SEQUENCE LISTING (30 sequences)

SEQ ID NO: 1 - DNA, 20 bp, Artificial sequence
  primer
ccaggcactcgtcctcggtt

SEQ ID NO: 2 - DNA, 48 bp, Artificial sequence
  primer
aggctagtgcaggactataaagaccagttctcctaaaaataacgtgtc

SEQ ID NO: 3 - DNA, 48 bp, Artificial sequence
  primer
gacacgttatttttaggagaactggtctttatagtcctgcactagcct

SEQ ID NO: 4 - DNA, 20 bp, Artificial sequence
  primer
tccatcgtggccaccgatcc

SEQ ID NO: 5 - DNA, 25 bp, Artificial sequence
  primer
cgggatccccaccggcgtactcgtg

SEQ ID NO: 6 - DNA, 28 bp, Artificial sequence
  primer
ccacggatccttccaatgctattggttg

SEQ ID NO: 7 - DNA, 27 bp, Artificial sequence
  primer
tgcgaggtcactttttcgctgttcagg

SEQ ID NO: 8 - DNA, 27 bp, Artificial sequence
  primer
gtcgataagctcttgctttttcgcagg

SEQ ID NO: 9 - DNA, 4394 bp, Corynebacterium glutamicum
  CDS (443)..(4213)
gtcgacaagcaaaatcgaagcggcagcacgccgcgtcggagccttaaacgccatcgccgccatccctgatggtttcaatcatcaagtcggtgaacgcgggcgcaacctgtcatccggacagcgccaactgatcgcgctggcgcgcgccgaactcatcgagccttccatcatgcttctcgacgaagccacctccaccctcgaccccgccaccgaagccgttatcctcaacgcctccgatcgagtcactaagggacgcaccagcatcatcgtcgcgcaccgcttggcaaccgctaaaagggccgaccgtattcttgttgttgaacaaggacgtatcattgaggacggatctcacgacgcgttgttgtctgctaacggcacctacgcccgcatgtggcatttaatggcctgacacgttatttttaggagaactgtcaacaaattaatgctacaactggggcttaggcataatcagccaacgaccaacgttacagtggataaaataaagctcaataaaccctcaagaagcaaggaaaagaggcgagtacctgccgtgagcagcgctagtactttcggccagaatgcgtggctggtagacgagatgttccagcagttccagaaggaccccaagtccgtggacaaggaatggagagaactctttgaggcgcaggggggaccaaatgctacccccgctacaacagaagcacagccttcagcgcccaaggagtctgcgaaaccagcaccaaaggctgcccctgcagccaaggcagcaccgcgcgtagaaaccaagccggccgccaagaccgcccctaaggccaaggagtcctcagtgccacagcaacctaagcttccggagccaggacaaaccccaatcaggggtattttcaagtccatcgcgaagaacatggatatctccctggaaatcccaaccgcaacctcggttcgcgatatgccagctcgcctcatgttcgaaaaccgcgcgatggtcaacgatcagctcaagcgcacccgcggtggcaagatctccttcacccacatcattggctacgccatggtgaaggcagtcatggctcacccggacatgaacaactcctacgacgtcatcgacggcaagccaaccctgatcgtgcctgagcacatcaacctgggccttgccatcgaccttcctcagaaggacggctcccgcgcacttgtcgtagcagccatcaaggaaaccgagaagatgaacttctccgagttcctcgcagcatacgaagacatcgtgacacgctcccgcaagggcaagctcaccatggatgactaccagggcgttaccgtttccttgaccaacccaggtggcatcggtacccgccactctgtcccacgtctgaccaagggccagggcaccatcatcggtgtcggttccatggattacccagcagagttccagggcgcttccgaagaccgccttgcagagctcggcgttggaaagcttgtcaccatcacctccacctacgatcaccgcgtgatccagggtgctgtgtccggtgaattcctgcgtaccatgtctcgcctgctcaccgatgattccttctgggatgagatcttcgacgcaatgaacgttccttacaccccaatgcgttgggcacaggacgttccaaacaccggtgttgataagaacacccgcgtcatgcagctcattgaggcataccgctcccgtggacacctcatcgctgacaccaacccactttcatgggttcagcctggcatgccagttccagaccaccgcgacctcgacatcgagacccacagcctgaccatctgggatctggaccgtaccttcagcgtcggtggcttcggcggcaaggagaccatgaccctgcgcgaggtactgtcccgcctgcgcgctgcctacaccttgaaggtcggctccgaatacacccacatcctggaccgcgacgagcgcacctggctgcaggaccgcctcgaagccggaatgccaaagccaacccaggcagagcagaagtacatcctgcagaagctgaacgccgcagaggctttcgagaacttcctgcagaccaagtacgtcggccagaagcgcttctccctcgaaggtgcagaagctctcatcccactgatggactccgccatcgacaccgccgcaggccagggcctcgacgaagttgtcatcggtatgccacaccgtggtcgcctcaacgtgctgttcaacatcgtgggcaagccactggcatccatcttcaacgagtttgaaggccaaatggagcagggccagatcggtggctccggtgacgtgaagtaccacctcggttccgaaggccagcacctgcagatgttcggcgacggcgagatcaaggtctccctgactgctaacccgtcccacctggaagctgttaacccagtgatggaaggtatcgtccgcgcaaagcaggactacctggacaagggcgtagacggcaagactgttgtgccactgctgctccacggtgacgctgcattcgcaggcctgggcatcgtgccagaaaccatcaacctggctaagctgcgtggctacgacgtcggaggcaccatccacatcgtggtgaacaaccagatcggcttcaccaccaccccagactccagccgctccatgcactacgcaaccgactacgccaaggcattcggctgcccagtcttccacgtcaatggtgatgacccagaggcagttgtctgggttggccagctggcaaccgagtaccgtcgtcgcttcggcaaggacgtcttcatcgacctcgtttgctaccgcctccgcggccacaacgaagctgatgatccttccatgacccagccaaagatgtatgagctcatcaccggccgcgagaccgttcgtgctcagtacaccgaagacctgctcggacgtggagacctctccaacgaagatgcagaagcagtcgtccgcgacttccacgaccagatggaatctgtgttcaacgaagtcaaggaaggcggcaagaagcaggctgaggcacagaccggcatcaccggctcccagaagcttccacacggccttgagaccaacatctcccgtgaagagctcctggaactgggacaggctttcgccaacaccccagaaggcttcaactaccacccacgtgtggctccagttgctaagaagcgcgtctcctctgtcaccgaaggtggcatcgactgggcatggggcgagctcctcgccttcggttccctggctaactccggccgcttggttcgccttgcaggtgaagattcccgccgcggtaccttcacccagcgccacgcagttgccatcgacccagcgaccgctgaagagttcaacccactccacgagcttgcacagtccaagggcaacaacggtaagttcctggtctacaactccgcactgaccgagtacgcaggcatgggcttcgagtacggctactccgtaggaaacgaagactccgtcgttgcatgggaagcacagttcggcgacttcgccaacggcgctcagaccatcatcgatgagtacgtctcctcaggcgaagctaagtggggccagacctccaagctgatccttctgctgcctcacggctacgaaggccagggcccagaccactcttccgcacgtatcgagcgcttcctgcagctgtgcgctgagggttccatgactgttgctcagccatccaccccagcaaaccacttccacctgctgcgtcgtcacgctctgtccgacctgaagcgtccactggttatcttcaccccgaagtccatgctgcgtaacaaggctgctgcctccgcaccagaagacttcactgaggtcaccaagttccaatccgtgatcgacgatccaaacgttgcagatgcagccaaggtgaagaaggtcatgctggtctccggcaagctgtactacgaattggcaaagcgcaaggagaaggacggacgcgacgacatcgcgatcgttcgtatcgaaatgctccacccaattccgttcaaccgcatctccgaggctcttgccggctaccctaacgctgaggaagtcctcttcgttcaggatgagccagcaaaccagggcccatggccgttctaccaggagcacctcccagagctgatcccgaacatgccaaagatgcgccgcgtttcccgccgcgctcagtcctccaccgcaactggtgttgctaaggtgcaccagctggaggagaagcagcttatcgacgaggctttcgaggcttaagtctttatagtcctgcactagcctagagggccttatgcagtgtgaatcacacagcataaggccctttttgctgccgtggttgcctaaggtggaaggcatgaaacgaatctgtgcggtcacgatctcttcagtacttttgctaagtggctgctcctccacttccaccacgcagctcgag

SEQ ID NO: 10 - protein, 1257 aa, Corynebacterium glutamicum
MLQLGLRHNQPTTNVTVDKIKLNKPSRSKEKRRVPAVSSASTFGQNAWLVDEMFQQFQKDPKSVDKEWRELFEAQGGPNATPATTEAQPSAPKESAKPAPKAAPAAKAAPRVETKPAAKTAPKAKESSVPQQPKLPEPGQTPIRGIFKSIAKNMDISLEIPTATSVRDMPARLMFENRAMVNDQLKRTRGGKISFTHIIGYAMVKAVMAHPDMNNSYDVIDGKPTLIVPEHINLGLAIDLPQKDGSRALVVAAIKETEKMNFSEFLAAYEDIVTRSRKGKLTMDDYQGVTVSLTNPGGIGTRHSVPRLTKGQGTIIGVGSMDYPAEFQGASEDRLAELGVGKLVTITSTYDHRVIQGAVSGEFLRTMSRLLTDDSFWDEIFDAMNVPYTPMRWAQDVPNTGVDKNTRVMQLIEAYRSRGHLIADTNPLSWVQPGMPVPDHRDLDIETHSLTIWDLDRTFSVGGFGGKETMTLREVLSRLRAAYTLKVGSEYTHILDRDERTWLQDRLEAGMPKPTQAEQKYILQKLNAAEAFENFLQTKYVGQKRFSLEGAEALIPLMDSAIDTAAGQGLDEVVIGMPHRGRLNVLFNIVGKPLASIFNEFEGQMEQGQIGGSGDVKYHLGSEGQHLQMFGDGEIKVSLTANPSHLEAVNPVMEGIVRAKQDYLDKGVDGKTVVPLLLHGDAAFAGLGIVPETINLAKLRGYDVGGTIHIVVNNQIGFTTTPDSSRSMHYATDYAKAFGCPVFHVNGDDPEAVVWVGQLATEYRRRFGKDVFIDLVCYRLRGHNEADDPSMTQPKMYELITGRETVRAQYTEDLLGRGDLSNEDAEAVVRDFHDQMESVFNEVKEGGKKQAEAQTGITGSQKLPHGLETNISREELLELGQAFANTPEGFNYHPRVAPVAKKRVSSVTEGGIDWAWGELLAFGSLANSGRLVRLAGEDSRRGTFTQRHAVAIDPATAEEFNPLHELAQSKGNNGKFLVYNSALTEYAGMGFEYGYSVGNEDSVVAWEAQFGDFANGAQTIIDEYVSSGEAKWGQTSKLILLLPHGYEGQGPDHSSARIERFLQLCAEGSMTVAQPSTPANHFHLLRRHALSDLKRPLVIFTPKSMLRNKAAASAPEDFTEVTKFQSVIDDPNVADAAKVKKVMLVSGKLYYELAKRKEKDGRDDIAIVRIEMLHPIPFNRISEALAGYPNAEEVLFVQDEPANQGPWPFYQEHLPELIPNMPKMRRVSRRAQSSTATGVAKVHQLEEKQLIDEAFEA

SEQ ID NO: 11 - DNA, 1471 bp, Corynebacterium glutamicum
  CDS (562)..(1182)
gtcgataagctcttgctttttcgcaggccccgcaaggtaacgtccggctaccgcgcgctggtacacgcgacgcacgttcgtatcgaccaccggcacgcgctgcccaaaatgaaacgccgcgaccgcgcgcgccgtgtaatcaccgatccccggcaacgcgagcagcgcctccaccgtatccggcacctcgccggcatgcttttcgacgatcacctccgcacattccttcaacctcagcgccctacgtggatagcccaacttgccccacgaccgcaaaatctcatcggtgctcgcattcgcgaaatcttccggagtgggccatttttccatccactcacgccaaatcggctcgactcgcgcgacgggagtttgttggctcatcacctctgaaaggagaattccccatgctgaagtattgggatcacgccacgcaagatcgcgggcatttgctctaaaccacacgagcagggctgtttgaaaagctgtaaatgacatgacctaaatgattgtactgactggcacattaggtcatacgtcacaccgagtggaagaatagagcttatgcctttgcgtaatgttgatagaactccgcccgcagtatgggaagcactgcttgccggaaacgaaagattcatcagtttcaacgaagatcgaccaaaccaggacgccccgcgcagaaaagaacttcgcaatgggcaaacgcctgcagctgttgtcatttcctgttcagattctcgagtgccagttgaatttatttttgacgtcggtctcggtgacctctttgttgtccgtactgccggagaaatcctcgaccaagcagtgcttgcatccatcgaatacgccactgaatccatcggcgttccattggttatcgtcatgggccacgaatcctgtggtgcagttgcagcaactgcagcagcacttgaaggcggtgcacttcccggaggctaccaacgagttttggtagaaaaggttgcaccatccattctagaagccaaggcagagggcctgagctccatcaaggaattcgaggaacaccacgttgtggcaacggtaaaccaactgttgtcccgttctccagagattcatcagaaggtcgaaaccggcgagttgggaatcattggtttgcgctaccgactctctgacggtcgtactgaacctgtaattagcaagaacgtgggttagttttcggtctgagattgcctatgatcggtgatttgggggccgtgggcagtgtggtttggtagttcgggggactcaagttagagaaaccagacaggcgtgccaagactctgggtttttccaggttttggaacgcctgtccggtttagaggtttagagattgagaaccggttgggaaccggacacaggtgccagaagttcggctttttaggtaatcttggcacgccagtttgatcgctgtggaaactatcacaattcccgtcctgaacagcgaaaaagtgacctcgca

SEQ ID NO: 12 - protein, 207 aa, Corynebacterium glutamicum
MPLRNVDRTPPAVWEALLAGNERFISFNEDRPNQDAPRRKELRNGQTPAAVVISCSDSRVPVEFIFDVGLGDLFVVRTAGEILDQAVLASIEYATESIGVPLVIVMGHESCGAVAATAAALEGGALPGGYQRVLVEKVAPSILEAKAEGLSSIKEFEEHHVVATVNQLLSRSPEIHQKVETGELGIIGLRYRLSDGRTEPVISKNVG

SEQ ID NO: 13 - DNA, 624 bp, Corynebacterium glutamicum
  CDS (1)..(621)
atgcctttgcgtaatgttgatagaactccgcccgcagtatgggaagcattgcttgccggaaacgaaagattcatcagtttcaacgaagatcgaccaaaccaggacgccccgcgcagaagagaacttcgcaatggacaaacgcctgcagctgttgttatttcctgttcagattctcgagtgccagttgagattatttttgacgtcggtctcggtgacctctttgttgtccgtactgccggagaaatcctcgaccaagcagtgcttgcgtccatcgaatacgccactgaatccatcggcgttccattggttatcgtcatgggccacgaatcctgtggtgcagttgcagcaactgcagcagcacttgaaggcggtgcacttcccggaggctaccaacgagttttggttgaaaaggttgcaccatccattctagaagccaaggcagagggcctgagctccatcaaggaattcgaggaacaccacgttgtggcaacggtaaaccaactgttgtcccgttctccagagattcatcagaaggtcgaaaccggtgagttgggaatcattggtttgcgctaccgactctctgacggtcgtactgaacctgtaattagcaagaacgtgggttag

SEQ ID NO: 14 - protein, 207 aa, Corynebacterium glutamicum
MPLRNVDRTPPAVWEALLAGNERFISFNEDRPNQDAPRRRELRNGQTPAAVVISCSDSRVPVEIIFDVGLGDLFVVRTAGEILDQAVLASIEYATESIGVPLVIVMGHESCGAVAATAAALEGGALPGGYQRVLVEKVAPSILEAKAEGLSSIKEFEEHHVVATVNQLLSRSPEIHQKVETGELGIIGLRYRLSDGRTEPVISKNVG

SEQ ID NO: 15 - DNA, 2367 bp, Corynebacterium glutamicum
  CDS (1)..(2364)
atgtctacagattactcatcaccagcatatttgcaaaaagttgataagtactggcgtgctgccaactatttatcagttggtcaactttatttaaaagataatcctttattacaacggccattaaaggctagtgacgttaaggttcacccaatcggtcactggggcacgattgccggccaaaacttcatctatgcgcatcttaaccgggtcatcaacaagtacggtttgaagatgttctacgttgaaggtccaggtcatggtggccaagtgatggtctccaactcataccttgatgggacttacacggatatttatcctgaaattacgcaggatgttgaagggatgcaaaaactcttcaagcaattctcattcccaggtggcgtggcttcccatgctgctcctgaaacaccaggctcaatccacgaaggtggcgaacttggttactcaatttcacacggtgttggggcaatccttgacaaccctgatgaaatcgccgcagtcgttgttggtgatggggaatccgaaaccggcccattagcaacttcatggcaatcaacgaagttcatcaacccaatcaacgatggggcagtgttaccaatcttgaaccttaacggctttaagatttctaacccaacgatttttggtcggacttctgatgaaaagatcaagcaatacttcgaaagcatgaactgggaaccaatctttgttgaaggtgacgatcctgaaaaggttcacccagctttagctaaggccatggatgaagccgtcgaaaagatcaaagccattcaaaagaacgctcgtgaaaacgatgacgctactttaccagtatggccgatgatcgtcttccgcgcacctaagggctggactggtcctaagtcatgggatggcgacaagatcgaaggttcattccgagctcaccaaattccaattcctgttgaccaaaccgacatggaacatgccgatgcgttagttgactggttggaatcatatcaaccaaaggaactcttcaatgaagatggttctttgaaggatgatatcaaagaaattatcccaactggcgatgcacggatggccgctaacccaatcactaatggtggggttgatccaaaggccttgaacttacctaacttccgtgattacgccgttgatacgtctaagcatggtgccaacgttaagcaagatatgatcgtttggtcagactacttgcgtgatgttatcaagaagaacccagataacttccggttatttggccctgatgaaaccatgtcaaaccggttatatggtgtctttgaaaccactaaccgtcaatggatggaagatattcacccagatagtgaccaatacgaagcacctgctggccgggtcttggatgctcaattatctgaacaccaagctgaaggttggttagaaggttacgtcttaactggtcgtcatggcttgtttgcaagttacgaagccttcttacgggttgtcgactcaatgttgacgcaacacttcaagtggttacgtaaggccaacgaacttgactggcggaagaagtacccgtcactcaacattatcgcggcttcaactgtgttccaacaagaccataatgggtacacccaccaagatccaggtgccttgactcatttggctgaaaagaagcctgaatatatccgcgaatatttaccagccgacgccaactccttgttagctgttggggacgtcatcttccgtagccaagaaaagatcaactacgtggttacgtcgaagcacccacgtcaacaatggttcagcattgaagaagctaagcaattagttgacaacggtcttggtatcattgactgggcaagcacggaccaaggtagcgaaccagatatcgtgtttgctgctgccggaacggaaccaacgcttgaaacgttggctgcaatccaattgctccatgatagcttcccagacatgaagattcgtttcgtgaacgtggtcgacatcttgaagttacgtagccctgaaaaggaccctcgtggcttgtcagatgctgaatttgaccattacttcactaaggacaaaccagttgtcttcgccttccatggttacgaagacctggttcgtgacatcttctttgatcgtcacaaccacaacttacacgtgcatggctaccgtgaaaatggtgacattacgacaccattcgatgtccgggtcatgaaccaaatggaccgtttcgacttagcaaaatctgcaattgcggcgcaaccagcaatggaaaacaccggtgcagcctttgttcaagacatggataacatgcttgcaaaacacaacgcatacatccgtgacgccggaaccgacttgccagaagttaacgactggcaatggaaaggtttgaaataa

SEQ ID NO: 16 - protein, 788 aa, Corynebacterium glutamicum
MSTDYSSPAYLQKVDKYWRAANYLSVGQLYLKDNPLLQRPLKASDVKVHPIGHWGTIAGQNFIYAHLNRVINKYGLKMFYVEGPGHGGQVMVSNSYLDGTYTDIYPEITQDVEGMQKLFKQFSFPGGVASHAAPETPGSIHEGGELGYSISHGVGAILDNPDEIAAVVVGDGESETGPLATSWQSTKFINPINDGAVLPILNLNGFKISNPTIFGRTSDEKIKQYFESMNWEPIFVEGDDPEKVHPALAKAMDEAVEKIKAIQKNARENDDATLPVWPMIVFRAPKGWTGPKSWDGDKIEGSFRAHQIPIPVDQTDMEHADALVDWLESYQPKELFNEDGSLKDDIKEIIPTGDARMAANPITNGGVDPKALNLPNFRDYAVDTSKHGANVKQDMIVWSDYLRDVIKKNPDNFRLFGPDETMSNRLYGVFETTNRQWMEDIHPDSDQYEAPAGRVLDAQLSEHQAEGWLEGYVLTGRHGLFASYEAFLRVVDSMLTQHFKWLRKANELDWRKKYPSLNIIAASTVFQQDHNGYTHQDPGALTHLAEKKPEYIREYLPADANSLLAVGDVIFRSQEKINYVVTSKHPRQQWFSIEEAKQLVDNGLGIIDWASTDQGSEPDIVFAAAGTEPTLETLAAIQLLHDSFPDMKIRFVNVVDILKLRSPEKDPRGLSDAEFDHYFTKDKPVVFAFHGYEDLVRDIFFDRHNHNLHVHGYRENGDITTPFDVRVMNQMDRFDLAKSAIAAQPAMENTGAAFVQDMDNMLAKHNAYIRDAGTDLPEVNDWQWKGLK

SEQ ID NO: 17 - DNA, 2367 bp, Lactobacillus plantarum
  CDS (1)..(2364)
atgacaacagattactcatcaccagcatatttgcaaaaagttgataagtactggcgtgctgccaactacttatcagttggtcaactttatttaaaagataatccactattacaacggccattgaaggccagtgacgttaaggttcatccaattggtcactgggggacgattgccggtcaaaactttatctatgctcatcttaaccgggtcatcaacaagtacggtttgaagatgttctacgttgaaggtccaggtcatggtggtcaagtgatggtttcaaactcttaccttgacggtacttacaccgatatttatccagaaattacgcaggatgttgaagggatgcaaaagctcttcaagcaattctcattcccaggtggggttgcttcccatgcggcacctgaaacacccggttcaatccacgaaggtggcgaacttggttactcaatttcacacggggttggggcaattcttgacaatcctgacgaaatcgccgcggttgttgttggtgatggggaatccgaaacgggtccattagcaacttcatggcaatcaacgaagttcattaacccaatcaacgacggggctgttttaccaatcttgaacttaaatggttttaagatttctaatccaacgatttttggtcggacttctgatgctaagattaaggaatacttcgaaagcatgaattgggaaccaatcttcgttgaaggtgacgatcctgaaaaggttcacccagccttagctaaggccatggatgaagccgttgaaaagatcaaggcaatccagaagcatgctcgcgaaaataacgatgcaacattgccagtatggccaatgatcgtcttccgcgcacctaagggctggactggtccgaagtcatgggacggtgataagatcgaaggttcattccgtgctcatcaaattccgattcctgttgatcaaaatgacatggaacatgcggatgctttagttgattggctcgaatcatatcaaccaaaagaactcttcaatgaagatggctctttgaaggatgatattaaagaaattattcctactggggacagtcggatggctgctaacccaatcaccaatggtggggtcgatccgaaagccttgaacttaccaaacttccgtgattatgcggtcgatacgtccaaagaaggcgcgaatgttaagcaagatatgatcgtttggtcagactatttgcgggatgtcatcaagaaaaatcctgataacttccggttgttcggacctgatgaaaccatgtctaaccgtttatatggtgtcttcgaaaccactaatcgtcaatggatggaagacattcatccagatagtgaccaatatgaagcaccagctggccgggtcttagatgctcagttatctgaacaccaagctgaaggttggttagaaggttacgtcttaactggacgtcatgggttatttgccagttatgaagccttcctacgcgttgtggactcaatgttgacgcaacacttcaagtggttacgtaaagccaatgaacttgattggcgtaaaaagtacccatcacttaacattatcgcggcttcaactgtattccaacaagaccataatggttatacccaccaagatccaggtgcattaactcatttggccgaaaagaaaccagaatacattcgtgaatatttaccagccgatgccaacacgttattagctgtcggtgacgtcattttccggagccaagaaaagatcaactacgtggttacgtcaaaacacccacgtcaacaatggttcagcattgaagaagctaagcaattagttgacaatggtcttggtatcattgattgggcaagtacggaccaaggtagcgaaccagacattgtctttgcagctgctgggacggaaccaacgcttgaaacgttggctgccatccaattactacacgacagtttcccagagatgaagattcgtttcgtgaacgtggtcgacatcttgaagttacgtagtcctgaaaaggatccgcggggcttgtcagatgctgagtttgaccattactttactaaggacaaaccagtggtctttgctttccacggttacgaagacttagttcgtgacatcttctttgatcgtcacaaccataacttatacgtccacggttaccgtgaaaatggtgatattaccacaccattcgacgtacgggtcatgaaccagatggaccgcttcgacttagctaagtcggcaattgcggcgcaaccagcaatggaaaacactggtgcggccttcgttcaatccatggataatatgcttgctaaacacaatgcctatatccgggatgccggaactgacttgccagaagttaatgattggcaatggaagggtttaaaataa

SEQ ID NO: 18 - protein, 788 aa, Lactobacillus plantarum [sequence omitted]

SEQ ID NO: 19 - DNA, 2660 bp, Bifidobacterium lactis
  CDS (128)..(2602)
aggtcagcgtattcgcgtaacataatcagcgatcgggcacggagaccggcctgcaggacagcgccgaagcccgtgcccaacggaataaacaaatcgcacatttatgtgcaggagtacaggagcacacatgactaatcctgttattggtaccccatggcagaagctggatcgtccggtttccgaagaggccatcgaaggcatggacaagtactggcgcgtcgccaactacatgtctatcggccagatctacctgcgtagcaacccgctgatgaaggagcccttcacccgcgatgacgtgaagcaccgtctggtcggccactggggcaccaccccgggcctgaacttccttctcgcccacatcaaccgcctgatcgccgatcaccagcagaacaccgtgttcatcatgggtcctggccacggcggccctgcaggtaccgctcagtcctacatcgacggcacctacaccgagtactacccgaacatcaccaaggacgaagctggcctgcagaagttcttccgccagttctcctacccgggtggcattccttcccacttcgctccggagacgccgggctccatccacgaaggcggcgagctgggctacgccctgtcgcacgcctacggcgcgatcatggacaacccgagcctcttcgtcccgtgcatcatcggtgacggcgaagccgagaccggccctctggccaccggctggcagtccaacaagctcgtcaacccgcgcaccgacggcatcgtcctgccgatcctgcacctcaacggctacaagatcgccaacccgacgatcctcgcccgcatctccgacgaggagctgcacgacttcttccgcggcatgggttaccacccgtacgagttcgtcgccggcttcgacaacgaggatcacctgtcgatccaccgtcgcttcgccgagctcttcgagaccatcttcgacgagatctgcgatatcaaggctgcggctcagaccgacgacatgacccgtccgttctacccgatgctcatcttccgcaccccgaagggctggacctgcccgaagttcatcgacggcaagaagaccgaaggctcctggcgtgcacaccaggtcccgctggcttccgcccgcgacaccgaggcccacttcgaagtcctcaagggctggatggaatcctacaagccggaggagctcttcaacgccgacggctccatcaaggaggacgtcaccgcattcatgcctaagggcgaactgcgcatcggcgccaacccgaatgccaacggcggccgcatccgcgaggatctgaagctccctgagctcgatcagtacgagatcaccggcgtcaaggaatacggccacggttggggccaggtcgaggctccgcgttccctcggcgcgtactgccgcgacatcatcaagaacaacccggattcgttccgcgtcttcggacctgacgagaccgcgtccaaccgtctgaacgcgacctacgaggtcaccaagaagcagtgggacaacggatacctctcggctctcgtcgacgagaacatggccgtcaccggccaggttgtcgagcagctctccgagcatcagtgcgaaggcttcctcgaggcctacctgctcaccggccgtcacggcatctggagctcctacgagtccttcgtgcacgtgatcgactccatgctgaaccagcatgcgaagtggctcgaggccaccgtccgcgagatcccgtggcgtaagccgatctcctcggtgaacctcctggtctcctcgcacgtgtggcgtcaggatcacaacggcttctcgcaccaggatccgggtgtgacctccgtcctgctgaacaagacgttcaacaacgaccacgtgacgaacatctacttcgcgaccgatgccaacatgctgctggccatcgccgagaagtgcttcaagtccaccaacaagatcaacgcaatcttcgccggcaagcagccggccgcgacgtggatcaccctcgacgaggtacgcgccgagctcgaggctggtgccgccgagtggaagtgggcttccaacgccaagagcaacgacgaggtccaggttgtcctcgccgccgccggcgacgtcccgacccaggagatcatggccgcttccgatgccctcaacaagatgggcatcaagttcaaggtcgtcaacgtcgtggacctcatcaagctgcagtcctcgaaggagaacgacgaggccatgtctgacgaggacttcgccgacctgttcaccgcggacaagccggtcctcttcgcctaccactcctatgcccaggacgttcgtggcctcatctacgaccgcccgaaccacgacaacttcaccgttgtcggatacaaggagcagggctccacgacgacgccgttcgacatggtgcgtgtcaacgacatggatcgctacgcccttcaggccaaggccctcgagctcatcgacgccgacaagtatgccgacaagatcaacgagctcaacgagttccgcaagaccgcgttccagttcgccgtcgacaatggctatgacattcctgagttcaccgattgggtgtacccggatgtcaaggtcgacgagacctccatgctctccgccaccgccgcgaccgccggcgacaacgagtgagcatagtctcatcgcttagccgatgaaaggcccgggtgtccgcacccgggccttt

SEQ ID NO: 20 - protein, 825 aa, Bifidobacterium lactis
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMDNPSLFVPCIIGDGEAETGPLATGWQSNKLVNPRTDGIVLPILHLNGYKIANPTILARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKEDVTAFMPKGELRIGANPNANGGRIREDLKLPELDQYEITGVKEYGHGWGQVEAPRSLGAYCRDIIKNNPDSFRVFGPDETASNRLNATYEVTKKQWDNGYLSALVDENMAVTGQVVEQLSEHQCEGFLEAYLLTGRHGIWSSYESFVHVIDSMLNQHAKWLEATVREIPWRKPISSVNLLVSSHVWRQDHNGFSHQDPGVTSVLLNKTFNNDHVTNIYFATDANMLLAIAEKCFKSTNKINAIFAGKQPAATWITLDEVRAELEAGAAEWKWASNAKSNDEVQVVLAAAGDVPTQEIMAASDALNKMGIKFKVVNVVDLIKLQSSKENDEAMSDEDFADLFTADKPVLFAYHSYAQDVRGLIYDRPNHDNFTVVGYKEQGSTTTPFDMVRVNDMDRYALQAKALELIDADKYADKINELNEFRKTAFQFAVDNGYDIPEFTDWVYPDVKVDETSMLSATAATAGDNE

SEQ ID NO: 21 - DNA, 1386 bp, Corynebacterium glutamicum
  CDS (1)..(1383)
atgtctgacacaccgacctcagctctgatcaccacggtcaaccgcagcttcgatggattcgatttggaagaagtagcagcagaccttggagttcggctcacctacctgcccgacgaagaactagaagtatccaaagttctcgcggcggacctcctcgctgaggggccagctctcatcatcggtgtaggaaacacgtttttcgacgcccaggtcgccgctgccctcggcgtcccagtgctactgctggtagacaagcaaggcaagcacgttgctcttgctcgcacccaggtaaacaatgccggcgcagttgttgcagcagcatttaccgctgaacaagagccaatgccggataagctgcgcaaggctgtgcgcaaccacagcaacctcgaaccagtcatgagcgccgaactctttgaaaactggctgctcaagcgcgcacgcgcagagcactcccacattgtgctgccagaaggtgacgacgaccgcatcttgatggctgcccaccagctgcttgatcaagacatctgtgacatcacgatcctgggcgatccagtaaagatcaaggagcgcgctaccgaacttggcctgcaccttaacactgcatacctggtcaatccgctgacagatcctcgcctggaggaattcgccgaacaattcgcggagctgcgcaagtcaaagagcgtcactatcgatgaagcccgcgaaatcatgaaggatatttcctacttcggcaccatgatggtccacaacggcgacgccgacggaatggtatccggtgcagcaaacaccaccgcacacaccattaagccaagcttccagatcatcaaaactgttccagaagcatccgtcgtttcttccatcttcctcatggtgctgcgcgggcgactgtgggcattcggcgactgtgctgttaacccgaacccaactgctgaacagcttggtgaaatcgccgttgtgtcagcaaaaactgcagcacaatttggcattgatcctcgcgtagccatcttgtcctactccactggcaactccggcggaggctcagatgtggatcgcgccatcgacgctcttgcagaagcacgccgacttaacccagaactatgcgtcgatggaccacttcagttcgacgccgccgtcgacccgggtgtggcgcgcaagaagatgccagactctgacgtcgctggccaggcaaatgtgtttatcttccctgacctggaagccggaaacatcggctacaaaactgcacaacgcaccggtcacgccctggcagttggtccgattctgcagggcctaaacaaaccagtcaacgacctttcccgtggcgcaacagtccctgacatcgtcaacacagtagccatcacagcaattcaggcaggaggacgcagctaa

SEQ ID NO: 22 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 23 - DNA, 2644 bp, Corynebacterium glutamicum
  CDS (1214)..(2641)
ttccaaagtctggcacacgaggtcctgagacttggaatcacgacgagcctgctccgaagctgcatcgtaatcaatgtcctcagggttcacgatcacctcgatggtgtcggaatgatcaagttccttcagttccaacggagtgaacttcgcctgagcaggtccacgacgaccaaaaacgtgcacttccttagcctgattcttagccaagctctcatagacattgtcagggatttcagtaactagcagctcatcgccagtcttcgccaaaatacgagcaacgtccaacgccacgttaccgacaccaacaaccgctaccttctcagcagaaagatcccagttgcgttcaaagttcggattgccatcatagaaaccaacgaactcgccagcgccccacgaaccttccagatcagaacctggaacccgaagatcctggtcgccagtagcgccagtggagaacacgatcgcgtcataaaactcacgcaactcctcaacagtgatgtccttgccgacctcaatgttgcccaagaaacgcagctgctccttgtccatcacattgtgcagggacttcacgatgcccttgatgcgagggtgatcaggcgcaacaccataacggatcaaaccgaaaggcgctggcatgcgttcaaaaagatcaatctgcacgtccgtgtcggatttcatcaacaaatcagacgcgtagattcctgctggacctgcaccgacaacggcaacacgcaaagggcgagacatataaagttcgattccttaaaggggttctaaaaaatgtggagtatgtgagcggggttccactagtagattcgactcctatcggggtgcgactgctaatggtgccctgctatcaaccctccatgatacgtggtaagtgcagactaataaaggccagtcggggagtattgggggctttgctgggggcagatttgtcacgctgcgcgctttcatagaccccattaatgtggggtgaagagctgtaaagtaccgctaaaaactttgcaaagggtgcttcgcaacttgtaaccgctccgtattgttttctacggcaataagcatttgtgctgctcaaagcgtggaattgagatcggtttgaaaattacaaaataaaactttgcaaaccgggctgtacgcaaggcggacgaacgctaaactatgtaagaaatcacaacttcccctcagtagtgccaggaggcacaagcctgaagtgtcatcaatgagaaggttcaggctgaaactagaaaggcgatgtatgtctgacacaccgacctcagctctgatcaccacggtcaaccgcagcttcgatggattcgatttggaagaagtagcagcagaccttggagttcggctcaccgacctgcccgacgaagaactagaagtatccaaagttctcgcggcggacctcctcgctgaggggccagctctcatcatcggtgtaggaaacacgtttttcgacgcccaggtcgccgctgccctcggcgtcccagtgctactgctggtagacaagcaaggcaagcacgttgctcttgctcgcacccaggtaaacaatgccggcgcagttgttgcagcagcatttaccgctgaacaagagccaatgccggataagctgcgcaaggctgtgcgcaaccacagcaacctcgaaccagtcatgagcgccgaactctttgaaaactggctgctcaagcgcgcacgcgcagagcactcccacattgtgctgccagaaggtgacgacgaccgcatcttgatggctgcccaccagctgcttgatcaagacatctgtgacatcacgatcctgggcgatccagtacagatcaaggagcgcgctaccgaacttggcctgcaccttaacactgcatacctggtcaatccgctgacagatcctcgcctggaggaattcgccgaacaattcgcggagctgcgcaagtcaaagagcgtcactatcgatgaagcccgcgaaatcatgaaggatatttgctacttcggcaccatgatggtccacaacggcgacgccgacggaatggtatccggtgcagcaaacaccaccgcacacaccattaagccaagcttccagatcatcaaaactgttccagaagcatccgtcgtttcttccatcttcctcatggtgctgcgcgggcgactgtgggcattcggcgactgtgctgttaacccgaacccaactgctgaacagcttggtgaaatcgccgttgtgtcagcaaaaactgcagcacaatttggcattgatcctcgcgtagccatcttgtcctactccactggcaactccggcggaggctcagatgtggatcgcgccatcgacgctcttgcagaagcacgccgacttaacccagaactatgcgtcgatggaccacttcagttcgacgccgccgtcgacccgggtgtggcgcgcaagaagatgccagactctgacgtcgctggccaggcaaatgtgtttatcttccctgacctggaagccggaaacatcggctacaaaactgcacaacgcaccggtcacgccctggcagttggtccgattctgcagggcctaaacaaaccagtcaacgacctttcccgtggcgcaacagtccctgacatcgtcaacacagtagccatcacagcaattcaggcaggaggacgcagctaa

SEQ ID NO: 24 - protein, 476 aa, Corynebacterium glutamicum
VSSMRRFRLKLERRCMSDTPTSALITTVNRSFDGFDLEEVAADLGVRLTDLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVQIKERATELGLHLNTAYLVNPLTDPRLEEFAEQFAELRKSKSVTIDEAREIMKDICYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 25 - DNA, 37 bp, Artificial sequence
  primer
cggggtacctataaagttcgattccttaaaggggttc

SEQ ID NO: 26 - DNA, 38 bp, Artificial sequence
  primer
cgcggatccgtgccaatgccattagctgcgtcctcctg

SEQ ID NO: 27 - DNA, 1063 bp, Escherichia coli
  CDS (201)..(860)
cccgcttcgacctgcaaatctatcccacgaagcgcctgaacgccgcctggataggtttttttaagctgttgaagttccagtgcaatggtcataaatttttacttaccttacgttcttacactttatctgtggtttaaatcgtcccggagttgccctatattagccaaacgtaattatttggttacaggtcgttaacctccatgaaagacatagatacactcatcagcaacaatgcactatggtcaaaaatgctggtggaagaggatcccgggttttttgagaaactggcacaagcgcaaaaaccgcgctttctatggattggatgttccgacagtcgcgttcctgcagaacgtttaaccggtcttgagccgggcgaactctttgttcaccgtaatgttgctaacctggtcattcacactgacctgaactgcctttccgtggttcagtatgcagtggatgtactcgaagttgaacacattattatctgtggccactacggttgcggcggcgtacaagccgcagttgaaaacccggaactggggcttatcaacaactggctgctgcatatccgcgatatctggttcaaacatagctcattgctcggcgaaatgccgcaagagcgccgtctggataccttgtgtgaactgaacgtcatggaacaggtgtataacctgggccactccaccattatgcaatcagcgtggaaacgcgggcagaaagttaccattcacggctgggcctacggcattcacgacggcttgctgcgtgatctggatgttaccgccaccaaccgcgaaacccttgagcaacgttaccgtcacgggatttccaacctcaagctgaaacacgccaaccacaaataaaaatgccatgccggatgcaacacatccggcaacttcacacttactcgtccagcagaatcactttgccgatatacggcagatgacggtaacgctgtgcgtaatcaatgccgtaacccaccacaaactcatccgggatcgagaaaccgataaattctaccgggacgttcacttcacgacgggacggtttatccagcagcgta

SEQ ID NO: 28 - protein, 220 aa, Escherichia coli
MKDIDTLISNNALWSKMLVEEDPGFFEKLAQAQKPRFLWIGCSDSRVPAERLTGLEPGELFVHRNVANLVIHTDLNCLSVVQYAVDVLEVEHIIICGHYGCGGVQAAVENPELGLINNWLLHIRDIWFKHSSLLGEMPQERRLDTLCELNVMEQVYNLGHSTIMQSAWKRGQKVTIHGWAYGIHDGLLRDLDVTATNRETLEQRYRHGISNLKLKHANHK

SEQ ID NO: 29 - DNA, 1060 bp, Escherichia coli
  CDS (201)..(857)
gcaggttgggagacgtgcaacgcactggcggcacgggtgaagctgccatgttcagccacggcaagaaaataattgatatgtcgagagagcattcgcaacctataagtaaatccaatggaactcatcataaatgagacttttaccttatgacaatcggcgagtagtctgcctctcattccagagacagacagaggttaacggtgaaagagattattgatggattccttaaattccagcgcgaggcatttccgaagcgggaagccttgtttaaacagctggcgacacagcaaagcccgcgcacactttttatctcctgctccgacagccgtctggtccctgagctggtgacgcaacgtgagcctggcgatctgttcgttattcgcaacgcgggcaatatcgtcccttcctacgggccggaacccggtggcgtttctgcttcggtggagtatgccgtcgctgcgcttcgggtatctgacattgtgatttgtggtcattccaactgtggcgcgatgaccgccattgccagctgtcagtgcatggaccatatgcctgccgtctcccactggctgcgttatgccgattcagcccgcgtcgttaatgaggcgcgcccgcattccgatttaccgtcaaaagctgcggcgatggtacgtgaaaacgtcattgctcagttggctaatttgcaaactcatccatcggtgcgcctggcgctcgaagaggggcggatcgccctgcacggctgggtctacgacattgaaagcggcagcatcgcagcttttgacggcgcaacccgccagtttgtgccactggccgctaatcctcgcgtttgtgccataccgctacgccaaccgaccgcagcgtaaccttatttttaaaccatcaggagttccaccatgattcagtcacaaattaaccgcaatattcgtcttgatcttgccgatgccattttgctcagcaaagctaaaaaagatctctcatttgccgagattgccgacggcaccggtctggcagaagcctttgtaaccgcggctttgctgggtcagcaggcgcttcctgccgacgc

SEQ ID NO: 30 - protein, 219 aa, Escherichia coli
VKEIIDGFLKFQREAFPKREALFKQLATQQSPRTLFISCSDSRLVPELVTQREPGDLFVIRNAGNIVPSYGPEPGGVSASVEYAVAALRVSDIVICGHSNCGAMTAIASCQCMDHMPAVSHWLRYADSARVVNEARPHSDLPSKAAAMVRENVIAQLANLQTHPSVRLALEEGRIALHGWVYDIESGSIAAFDGATRQFVPLAANPRVCAIPLRQPTAA

(19) United States
(12) Patent Application Publication
(10) Pub. No.: US 2012/0237985 A1
(43) Pub. Date: Sep. 20, 2012

(54) METHOD FOR PRODUCING L-AMINO ACID

(76) Inventors: Takeshi Nagahiko, (US); Jun Nakamura, (US)

(21) Appl. No.: 13/404,105

(22) Filed: Feb. 24, 2012

Related U.S. Application Data
(63) Continuation of application No. PCT/JP10/62253, filed on Jul. 21, 2010.

(30) Foreign Application Priority Data
Aug. 25, 2009 (JP) 2009-194636

(51) Int. Cl. C12P 13/04; C12P 13/12; C12P 13/10; C12P 13/06; C12P 13/14; C12P 13/24

(52) U.S. Cl. 435/107; 435/106; 435/110; 435/114; 435/116; 435/113

(57) ABSTRACT

A method for producing an L-amino acid by culturing a coryneform bacterium having an L-amino acid-producing ability in a medium to produce and accumulate the L-amino acid in the medium or cells of the bacterium, and collecting the L-amino acid from the medium or cells, wherein said coryneform bacterium has been modified to enhance carbonic anhydrase activity.

This application is a Continuation of, and claims priority under 35 U.S.C. §120 to, International Application No. PCT/JP2010/062253, filed Jul. 21, 2010, and claims priority therethrough under 35 U.S.C. §119 to Japanese Patent Application No. 2009-194636, filed Aug. 25, 2009, the entireties of which are incorporated by reference herein. Also, the Sequence Listing filed electronically herewith is hereby incorporated by reference (File name: 2012-02-24T_US-475_Seq_List; File size: 128 KB; Date recorded: Feb. 24, 2012).

### BACKGROUND OF THE INVENTION

1. Technical Field

The present invention relates to a method for efficiently producing by fermentation an L-amino acid such as L-glutamic acid, L-glutamine, L-proline, L-arginine, L-asparagine, L-asparatic acid, L-lysine, L-methionine, L-threonine and L-isoleucine.

2. Background Art

L-Amino acids are industrially produced by fermentation mainly using L-amino acid-producing bacteria of the so-called coryneform bacteria belonging to the genus Brevibacterium, Corynebacterium or Microbacterium, or mutant strains thereof (refer to, for example, Akashi K. et al., Amino Acid Fermentation, Japan Scientific Societies Press, 195-215, 1986). Methods of producing an L-amino acid by fermentation using other bacterial strains have been reported, and include methods of using a microorganism belonging to the genus Bacillus, Streptomyces, Penicillium or the like (refer to, for example, U.S. Pat. No. 3,220,929), methods of using a microorganism belonging to the genus Pseudomonas, Arthrobacter, Serratia, Candida or the like (refer to, for example, U.S. Pat. No. 3,563,857), methods of using a microorganism belonging to the genus Bacillus, Pseudomonas, or Serratia, Aerobacter aerogenes (currently referred to as Enterobacter aerogenes) or the like (refer to, for example, Japanese Patent Publication (KOKOKU) No. 32-9393), methods of using a mutant strain of Escherichia coli (refer to, for example, Japanese Patent Laid-open (KOKAI) No. 5-244970), and so forth. In addition, methods for producing an L-amino acid using a microorganism belonging to the genus Klebsiella, Erwinia, Pantoea or Enterobacter have also been disclosed (refer to, for example, Japanese Patent Laid-open No. 2000-106869, Japanese Patent Laid-open No. 2000-189169, and Japanese Patent Laid-open No. 2000-189175).

Furthermore, various techniques have been disclosed of increasing L-amino acid-producing ability by enhancing an activity of an enzyme for biosynthesis of L-amino acid using recombinant DNA techniques. For example, it has been reported that introduction of a gene encoding a citrate synthase derived from Escherichia coli or Corynebacterium glutamicum into a Corynebacterium or Brevibacterium bacterium is effective for enhancement of L-glutamic acid-producing ability of the coryneform bacterium (for example, refer to Japanese Patent Publication No. 7-121228). Furthermore, it has also been reported that introduction of a citrate synthase gene derived from a coryneform bacterium into an enterobacterium belonging to the genus Enterobacter, Klebsiella, Serratia, Erwinia or Escherichia is effective for enhancement of L-glutamic acid-producing ability of the bacterium (refer to, for example, Japanese Patent Laid-open No. 2000-189175).

Carbonic anhydrase is an enzyme involved in the mutual conversion of carbon dioxide and bicarbonate radical. It has been reported that Escherichia coli has two kinds of carbonic anhydrases, i.e., Can (carbonic anhydrase 2) and CynT (carbonic anhydrase 1) (J. Biol. Chem, 267, 3731-3734, 1992 and Smith K. S., Ferry J. G, “Prokaryotic Carbonic Anhydrases”, FEMS Microbiol. Rev., 24(4):335-66, 2000). It has been elucidated that Can is a β type carbonic anhydrase, and is indispensable for growth of Escherichia coli under the usual atmospheric carbon dioxide partial pressure. Can and CynT are encoded by the yadF and cynT genes, respectively. It is also known that, in Corynebacterium glutamicum, β type and γ type carbonic anhydrases have been found, and the β type one mainly functions (refer to Appl. Microbiol. Biotechnol., 63, 592-601, 2004).

Carbonic anhydrase has been reported to be useful in the production of ethanol from a vegetable raw material containing lignocellulose during pretreatment of the raw material (refer to U.S. Published Patent Application No. 2008/0171370). Furthermore, it was reported that enhanced β-carbonic anhydrase of Corynebacterium bacteria did not increase the production amount of lysine (refer to Appl. Microbiol. Biotechnol., 63, 592-601, 2004), and effectiveness of enhancement of β-carbonic anhydrase on production of a substance and relation between the β-carbonic anhydrase activity and L-amino acid productivity are still unknown.

### SUMMARY OF THE INVENTION

An aspect of the present invention is to provide a novel method for producing by fermentation an L-amino acid, especially L-glutamic acid, L-glutamine, L-proline, L-arginine, L-asparagine, L-asparatic acid, L-lysine, L-methionine, L-threonine, or L-isoleucine.

By culturing a coryneform bacterium that has been imparted with an L-amino acid-producing ability and has been modified to enhance carbonic anhydrase (henceforth carbonic anhydrase refers to β-carbonic anhydrase unless specifically indicated) activity, an L-amino acid such as L-glutamic acid, L-glutamine, L-proline, L-arginine, L-asparagine, L-asparatic acid, L-lysine, L-methionine, L-threonine, and L-isoleucine can be efficiently produced.

It is an aspect of the present invention to provide a method for producing an L-amino acid, which comprises culturing a coryneform bacterium having an L-amino acid-producing ability in a medium to produce and accumulate the L-amino acid in the medium or cells of the bacterium, and collecting the L-amino acid from the medium or cells, wherein said coryneform bacterium has been modified to enhance carbonic anhydrase activity.

It is a further aspect of the present invention to provide the method as described above, wherein said carbonic anhydrase activity is enhanced by a method selected from the group consisting of increasing a copy number of a gene encoding carbonic anhydrase, modifying an expression control sequence of the gene, and combinations thereof.

It is a further aspect of the present invention to provide the method as described above, wherein the gene encoding the carbonic anhydrase is a DNA selected from the group consisting of:

(a) a DNA comprising the nucleotide sequence of the nucleotide numbers 562 to 1182 of SEQ ID NO: 11, or the nucleotide sequence of SEQ ID NO: 13, or

(b) a DNA that is able to hybridize with a complement of the nucleotide sequence of the nucleotide numbers 562 to 1182 of SEQ ID NO: 11, or the nucleotide sequence of SEQ ID NO: 13 under stringent conditions, and encodes a protein having carbonic anhydrase activity.

It is a further aspect of the present invention to provide the method as described above, wherein the bacterium has been further modified to impart D-xylose-5-phosphate phosphoketolase activity and/or fructose-6-phosphte phosphoketolase activity.

It is a further aspect of the present invention to provide the method as described above, wherein the bacterium has been further modified to enhance phosphotransacetylase activity.

It is a further aspect of the present invention to provide the method as described above, wherein the bacterium has been further modified to enhance pyruvate carboxylase activity.

It is a further aspect of the present invention to provide the method as described above, wherein the bacterium has been further modified to enhance phosphoenolpyruvate carboxylase activity.

It is a further aspect of the present invention to provide the method as described above, wherein the L-amino acid is selected from the group consisting of L-glutamic acid, L-glutmine, L-proline, L-arginine, L-leucine, and L-cysteine.

According to the production method of the present invention, L-amino acids such as L-glutamic acid, L-glutamine, L-proline, L-arginine, L-asparagine, L-asparatic acid, L-lysine, L-methionine, L-threonine, and L-isoleucine can be efficiently produced.

### BRIEF DESCRIPTION OF THE DRAWINGS

FIG. 1 shows the L-amino acid accumulation amount produced by a carbonic anhydrase (BCA)-enhanced strain.

### DESCRIPTION OF THE PREFERRED EMBODIMENTS

Hereafter, the present invention will be explained in detail.

<1> Coryneform Bacteria

The coryneform bacterium can have an L-amino acid-producing ability and be modified to enhance carbonic anhydrase activity. The coryneform bacterium can be a coryneform bacterium which already has an L-amino acid-producing ability, and is then modified so that carbonic anhydrase activity thereof is enhanced. Alternatively, the coryneform bacterium can be a coryneform bacterium which inherently has an L-amino acid-producing ability, or can be a coryneform bacterium which has been imparted with an L-amino acid-producing ability by breeding utilizing a mutation method, recombinant DNA technique, or the like. The “coryneform bacteria” can also include bacteria which have previously been classified into the genus Brevibacterium but have since been united into the genus Corynebacterium (Int. J. Syst. Bacteriol., 41:255-260, 1991), and bacteria belonging to the genus Brevibacterium, which are closely related to the genus Corynebacterium.

Examples of such coryneform bacteria include the following:

Corynebacterium acetoacidophilum

Corynebacterium acetoglutamicum

Corynebacterium alkanolyticum

Corynebacterium callunae

Corynebacterium glutamicum

Corynebacterium lilium

Corynebacterium melassecola

Corynebacterium thermoaminogenes (Corynebacterium efficiens)

Corynebacterium herculis

Brevibacterium divaricatum

Brevibacterium flavum

Brevibacterium immariophilum

Brevibacterium lactofermentum

Brevibacterium roseum

Brevibacterium saccharolyticum

Brevibacterium thiogenitalis

Corynebacterium ammoniagenes

Brevibacterium album

Brevibacterium cerinum

Microbacterium ammoniaphilum

Specific examples of these bacteria include the following strains:

Corynebacterium acetoacidophilum ATCC 13870

Corynebacterium acetoglutamicum ATCC 15806

Corynebacterium alkanolyticum ATCC 21511

Corynebacterium callunae ATCC 15991

Corynebacterium glutamicum ATCC 13020, ATCC 13032, ATCC 13060

Corynebacterium lilium ATCC 15990

Corynebacterium melassecola ATCC 17965

Corynebacterium thermoaminogenes AJ12340 (PERM BP-1539)

Corynebacterium herculis ATCC 13868

Brevibacterium divaricatum ATCC 14020

Brevibacterium flavum ATCC 13826, ATCC 14067

Brevibacterium immariophilum ATCC 14068

Brevibacterium lactofermentum ATCC 13869

Brevibacterium roseum ATCC 13825

Brevibacterium saccharolyticum ATCC 14066

Brevibacterium thiogenitalis ATCC 19240

Corynebacterium ammoniagenes ATCC 6871, ATCC 6872

Brevibacterium album ATCC 15111

Brevibacterium cerinum ATCC 15112

Microbacterium ammoniaphilum ATCC 15354

These strains are available from, for example, the American Type Culture Collection (Address: 12301 Parklawn Drive, Rockville, Md. 20852, P.O. Box 1549, Manassas, Va. 20108, United States of America). That is, registration numbers are given to each of the strains, and the strains can be ordered by using these registration numbers (refer to www.atcc.org/). The registration numbers of the strains are listed in the catalogue of the American Type Culture Collection. The AJ12340 strain was deposited on Oct. 27, 1987 at National Institute of Bioscience and Human Technology, Agency of Industrial Science and Technology, Ministry of Economy, Trade and Industry (currently the independent administrative agency, National Institute of Advanced Industrial Science and Technology, International Patent Organism Depositary, Tsukuba Central 6, 1-1, Higashi 1-Chome, Tsukuba-shi, Ibaraki-ken, 305-5466, Japan) with an accession number of FERM BP-1539 under the provisions of Budapest Treaty.

The phrase “L-amino acid-producing ability” can mean an ability of the coryneform bacterium to cause accumulation of an L-amino acid in a medium, when the bacterium is cultured in the medium. This L-amino acid-producing ability can be a property that a wild-type strain of the coryneform bacterium has, or a property that is imparted or enhanced by breeding.

Examples of the L-amino acid include L-lysine, L-glutamic acid, L-threonine, L-valine, L-leucine, L-isoleucine, L-serine, L-asparatic acid, L-asparagine, L-glutamine, L-arginine, L-cysteine (cystine), L-methionine, L-phenylalanine, L-tryptophan, L-tyrosine, L-glycine, L-alanine, L-proline, L-ornithine, L-citrulline, and L-homoserine. However, L-amino acids derived from oxalacetic acid and acetyl-CoA are particular examples, and L-glutamic acid, L-glutamine, L-proline, L-arginine, L-asparagine, L-asparatic acid, L-lysine, L-methionine, L-threonine, and L-isoleucine are more particular examples.

<1-1>Impartation of L-Amino Acid-Producing Ability

Hereafter, methods for imparting an L-amino acid-producing ability to a coryneform bacterium, and coryneform bacteria imparted with an L-amino acid-producing ability will be explained with reference to examples.

Examples of the method for imparting or enhancing L-glutamic acid-producing ability by breeding include, for example, a method of modifying a bacterium so that expression of a gene encoding an enzyme involved in the L-glutamic acid biosynthesis is enhanced. Examples of such an enzyme involved in the L-glutamic acid biosynthesis include, for example, glutamate dehydrogenase, glutamine synthetase, glutamate synthase, isocitrate dehydrogenase, aconitate hydratase, citrate synthase, phosphoenolpyruvate carboxylase, pyruvate carboxylase, pyruvate dehydrogenase, pyruvate kinase, phosphoenolpyruvate synthase, enolase, phosphoglyceromutase, phosphoglycerate kinase, glyceraldehydes-3-phosphate dehydrogenase, triosephosphate isomerase, fructose bisphosphate aldolase, phosphofructokinase, glucose phosphate isomerase, and so forth.

Examples of the method for enhancing expression of such genes as mentioned above include by introducing an amplification plasmid obtained by introducing a DNA fragment containing any of these genes into an appropriate plasmid, for example, a plasmid containing at least a gene responsible for replication and proliferation of the plasmid in a coryneform bacterium, increasing the copy number of any of these genes on a chromosome by conjugation, gene transfer, or the like, and introduction of a mutation into a promoter region of any of these genes (refer to International Patent Publication WO95/34672)

When the aforementioned amplification plasmid is used, or copy number is increased on the chromosome, the promoter for expressing these genes may be any kind of promoter so long as the chosen promoter functions in coryneform bacteria, and can be a promoter of the chosen gene. The expression amount of a gene can also be controlled by appropriately choosing a promoter. Examples of coryneform bacteria modified by such methods as mentioned above so that expression of a citrate synthase gene, phosphoenolpyruvate carboxylase gene and/or glutamate dehydrogenase gene is enhanced include the coryneform bacteria disclosed in WO00/18935 and so forth.

L-glutamic acid-producing ability can be imparted by decreasing or eliminating the activity of an enzyme that catalyzes a reaction which branches off from the L-glutamic acid biosynthesis pathway and produces a compound other than L-glutamic acid. Examples of such an enzyme include isocitrate lyase, α-ketoglutarate dehydrogenase, phosphate acetyltransferase, acetate kinase, acetohydroxy acid synthase, acetolactate synthase, formate acetyltransferase, lactate dehydrogenase, glutamate decarboxylase, 1-pyrroline dehydrogenase, and so forth.

In order to reduce or eliminate the activity of these enzymes, a mutation may be introduced into the gene encoding the enzyme on the chromosome by a usual mutagenesis method so that the intracellular activity of the enzyme is reduced or eliminated. Such introduction of a mutation can be achieved by, for example, using genetic recombination to eliminate the genes encoding the enzymes on the chromosome, or modifying an expression control sequence such as a promoter or the Shine-Dalgarno (SD) sequence. It can also be achieved by introducing a mutation resulting in an amino acid substitution (missense mutation), a stop codon (nonsense mutation), or a frame shift mutation which adds or deletes one or two nucleotides into regions encoding the enzymes on the chromosome, or partially deleting the genes (J. Biol. Chem., 272:8611-8617, 1997). The enzymatic activities can also be decreased or eliminated by constructing a gene encoding a mutant enzyme in which the coding region is deleted, and substituting it for a normal gene on a chromosome by homologous recombination or the like.

For example, in order to decrease the α-ketoglutarate dehydrogenase activity, the sucA (odhA) gene encoding the E1o subunit of the enzyme can be used.

The nucleotide sequence of the sucA gene and the amino acid sequence encoded thereby are shown as SEQ ID NOS: 9 and 10. For example, disruption of the sucA gene can be performed by the method described in the example described later using the primers of SEQ ID NOS: 1 to 6.

In addition, examples of strains with decreased α-ketoglutarate dehydrogenase activity include, for example, the following strains:

Brevibacterium lactofermentum AS strain (WO95/34672)

Brevibacterium lactofermentum AJ12821 (FERM BP-4172, French Patent No. 9401748)

Brevibacterium flavum AJ12822 (FERM BP-4173, French Patent No. 9401748)

Corynebacterium glutamicum AJ12823 (FERM BP-4174, French Patent No. 9401748)

Examples of other methods for imparting or enhancing L-glutamic acid-producing ability also include a method of imparting resistance to an organic acid analogue, a respiratory chain inhibitor, or the like, and a method of imparting sensitivity to a cell wall synthesis inhibitor. Examples of such methods include the method of imparting resistance to benzopyrones or naphthoquinones (Japanese Patent Laid-open No. 56-1889), the method of imparting resistance to HOQNO (Japanese Patent Laid-open No. 56-140895), the method of imparting resistance to α-ketomalonic acid (Japanese Patent Laid-open No. 57-2689), the method of imparting resistance to guanidine (Japanese Patent Laid-open No. 56-35981), the method of imparting sensitivity to penicillin (Japanese Patent Laid-open No. 4-88994), and so forth.

Specific examples of such resistant strains include the following strains:

Brevibacterium flavum AJ11355 (FERM P-5007, refer to Japanese Patent Laid-open No. 56-1889)

Corynebacterium glutamicum AJ11368 (FERM P-5020, refer to Japanese Patent Laid-open No. 56-1889)

Brevibacterium flavum AJ11217 (FERM P-4318, refer to Japanese Patent Laid-open No. 57-2689)

Corynebacterium glutamicum AJ11218 (FERM P-4319, refer to Japanese Patent Laid-open No. 57-2689)

Brevibacterium flavum AJ11564 (FERM BP-5472, refer to Japanese Patent Laid-open No. 56-140895)

Brevibacterium flavum AJ11439 (FERM BP-5136, refer to Japanese Patent Laid-open No. 56-35981)

Corynebacterium glutamicum H7684 (FERM BP-3004, refer to Japanese Patent Laid-open No. 04-88994)

Examples of method for imparting L-glutamine-producing ability include, for example, a method of modifying a bacterium so that expression of a gene encoding an enzyme involved in the L-glutamine biosynthesis is enhanced. Examples of the enzyme involved in the L-glutamine biosynthesis include, for example, glutamine synthetase and glutamate dehydrogenase (Japanese Patent Laid-open No. 2002-300887).

L-glutamine-producing ability can also be imparted by reducing or deleting the activity of an enzyme that catalyzes a reaction which branches off from the biosynthesis pathway of L-glutamine, and produces another compound. For example, it is conceivable to reduce intracellular glutaminase activity (Japanese Patent Laid-open No. 2004-187684).

Examples of methods for imparting or enhancing L-glutamine-producing ability also include imparting resistance to amino acid analogues and so forth. Specific examples include imparting 6-diazo-5-oxo-norleucine resistance (Japanese Patent Laid-open No. 3-232497), imparting purine analogue resistance and/or methionine sulfoxide resistance (Japanese Patent Laid-open No. 61-202694), imparting α-ketomalonic acid resistance (Japanese Patent Laid-open No. 56-151495), imparting resistance to a peptide containing glutamic acid (Japanese Patent Laid-open No. 2-186994), and so forth.

Specific examples of coryneform bacteria having L-glutamine-producing ability include the following strains:

Brevibacterium flavum AJ11573 (FERM P-5492, Japanese Patent Laid-open No. 56-151495)

Brevibacterium flavum AJ12210 (FERM P-8123, Japanese Patent Laid-open No. 61-202694)

Brevibacterium flavum AJ12212 (FERM P-8125, Japanese Patent Laid-open No. 61-202694)

Brevibacterium flavum AJ12418 (FERM-BP2205, Japanese Patent Laid-open No. 2-186994)

Brevibacterium flavum DH18 (FERM P-11116, Japanese Patent Laid-open No. 3-232497)

Corynebacterium melassecola DH344 (FERM P-11117, Japanese Patent Laid-open No. 3-232497)

Corynebacterium glutamicum AJ11574 (FERM P-5493, Japanese Patent Laid-open No. No. 56-151495)

Examples of method for imparting L-proline-producing ability include, for example, a method of modifying a bacterium so that expression of a gene encoding an enzyme involved in the L-proline biosynthesis is enhanced. Examples of the enzyme involved in the L-proline biosynthesis include, for example, glutamate-5-kinase, γ-glutamylphosphate reductase, and pyroline-5-carboxylate reductase.

L-proline-producing ability can be imparted by reducing or deleting an activity of an enzyme that catalyzes a reaction branching off from the biosynthesis pathway of L-proline to produce another compound. Examples include, for example, reducing intracellular ornithine aminotransferase activity.

Examples of a method for imparting L-arginine-producing ability include a method of modifying a bacterium so that expression of a gene encoding an enzyme involved in L-arginine biosynthesis is enhanced. Examples of L-arginine biosynthetic enzymes include N-acetylglutamyl phosphate reductase (argC), ornithine acetyltransferase (argJ), N-acetylglutamate kinase (argB), acetylornithine transaminase (argD), ornithine carbamoyl transferase (argF), argininosuccinate synthase (argG), argininosuccinate lyase (argH), and carbamoylphosphate synthase.

Another method for imparting L-arginine-producing ability can be a method of imparting resistance to an amino acid analogue or the like. Examples of bacteria obtained by such a method include coryneform bacteria exhibiting L-histidine, L-proline, L-threonine, L-isoleucine, L-methionine, or L-tryptophan auxotrophy in addition to the resistance to 2-thiazolealanine (Japanese Patent Laid-open No. 54-44096); coryneform bacteria resistant to ketomalonic acid, fluoromalonic acid, or monofluoroacetic acid (Japanese Patent Laid-open No. 57-18989); coryneform bacteria resistant to argininol (Japanese Patent Publication No. 62-24075); coryneform bacteria resistant to X-guanidine (X can be a derivative of fatty acid or aliphatic chain, Japanese Patent Laid-open No. 2-186995); coryneform bacteria resistant to arginine hydroxamate and 6-azauracil (Japanese Patent Laid-open No. 57-150381), and so forth.

In addition, since L-arginine, L-glutamine and L-proline have L-glutamic acid as a basic structure, a bacterium having ability to produce any of these amino acids may be bred by amplifying a gene encoding an enzyme that catalyses a reaction that generates any of the L-amino acids from L-glutamic acid in such L-glutamic acid-producing bacteria as mentioned above.

Further, the biosynthetic pathways of L-citrulline and L-ornithine are common to that of L-arginine, and therefore abilities to produce them can be imparted by increasing enzymatic activities of N-acetylglutamate synthase (argA), N-acetylglutamyl phosphate reductase (argC), ornithine acetyltransferase (argJ), N-acetylglutamate kinase (argB), acetylornithine transaminase (argD), and acetylornithine deacetylase (argE).

Coryneform bacteria having L-cysteine-producing ability include, for example, a coryneform bacterium with increased intracellular serine acetyltransferase activity by desensitizing the feedback inhibition by L-cysteine (Japanese Patent Laid-open No. 2002-233384).

Examples of method for imparting L-valine-producing ability include, for example, a method of modifying a bacterium so that expression of a gene encoding an enzyme involved in the L-valine biosynthesis is enhanced. Examples of enzymes involved in L-valine biosynthesis include enzymes encoded by the genes on the ilvBNC operon, that is, acetohydroxy acid synthetase encoded by ilvBN and isomero-reductase encoded by ilvC (WO00/50624). Since the ilvBNC operon is subject to expression regulation of the operon by L-valine and/or L-isoleucine and/or L-leucine, attenuation can be deleted to avoid suppressing expression by the produced L-valine.

A coryneform bacterium having L-valine-producing ability can also be obtained by decreasing or eliminating activity of at least one kind of enzyme involved in a metabolic pathway that decreases L-valine production. For example, the activity of threonine dehydratase which is involved in the L-leucine synthesis, or the activity of an enzyme involved in the D-panthothenate synthesis, can be decreased (WO00/50624).

Examples of methods for imparting L-valine-producing ability also include imparting resistance to an amino acid analogue or the like. Examples of bacteria obtained by such a method include, for example, mutant strains which are auxotrophic for L-isoleucine and L-methionine, and resistant to D-ribose, purine ribonucleoside or pyrimidine ribonucleoside, and have an ability to produce L-valine (FERM P-1841, FERM P-29, Japanese Patent Publication No. 53-025034), mutant strains resistant to polyketides (FERM P-1763, FERM P-1764, Japanese Patent Publication No. 06-065314), and mutant strains resistant to L-valine in a medium containing acetic acid as the sole carbon source and sensitive to pyruvic acid analogues (β-fluoropyruvic acid etc.) in a medium containing glucose as the sole carbon source (FERM BP-3006, BP-3007, Japanese Patent No. 3006929).

Examples of coryneform bacteria imparted with L-alanine-producing ability include, for example, coryneform bacteria lacking the HtATPase activity (Appl. Microbiol. Biotechnol., 2001 Nov., 57(4):534-40), coryneform bacteria in which aspartate β-decarboxylase gene is amplified (Japanese Patent Laid-open No. 07-163383), and so forth.

Examples of coryneform bacteria imparted with L-lysine-producing ability include lysine analogue resistant strains or metabolic regulation mutant strains having L-lysine-producing ability. Specific examples include S-(2-aminoethyl)-cysteine (henceforth abbreviated as “AEC”) resistant mutant strains (Brevibacterium lactofermentum AJ11082 (NRRL B-11470) strain etc., refer to Japanese Patent Publication Nos. 56-1914, 56-1915, 57-14157, 57-14158, 57-30474, 58-10075, 59-4993, 61-35840, 62-24074, 62-36673, 5-11958, 7-112437 and 7-112438); mutant strains requiring an amino acid such as L-homoserine for their growth (refer to Japanese Patent Publication Nos. 48-28078 and 56-6499); mutant strains showing resistance to AEC and further requiring an amino acid such as L-leucine, L-homoserine, L-proline, L-serine, L-arginine, L-alanine and L-valine (refer to U.S. Pat. Nos. 3,708,395 and 3,825,472); L-lysine-producing mutant strains showing resistance to DL-α-amino-ε-caprolactam, α-amino-lauryllactam, aspartic acid analogue, sulfa drug, quinoid and N-lauroylleucine; L-lysine-producing mutant strains showing resistance to oxaloacetate decarboxylase or a respiratory tract enzyme inhibitor (Japanese Patent Laid-open Nos. 50-53588, 50-31093, 52-102498, 53-9394, 53-86089, 55-9783, 55-9759, 56-32995, 56-39778, Japanese Patent Publication Nos. 53-43591 and 53-1833); L-lysine-producing mutant strains requiring inositol or acetic acid (Japanese Patent Laid-open Nos. 55-9784 and 56-8692); L-lysine-producing mutant strains that are susceptible to fluoropyruvic acid or a temperature of 34° C. or higher (Japanese Patent Laid-open Nos. 55-9783 and 53-86090); L-lysine-producing mutant strains of Brevibacterium or Corynebacterium bacteria showing resistance to ethylene glycol (U.S. Pat. No. 4,411,997), and so forth.

Furthermore, a coryneform bacterium imparted with L-lysine-producing ability can also be obtained by increasing activity of an L-lysine biosynthetic enzyme. Increase of activity of such an enzyme can be attained by increasing the copy number of a gene encoding the enzyme in cells, or by modifying an expression control sequence thereof.

Examples of genes encoding L-lysine biosynthetic enzymes include genes encoding enzymes of the diaminopimelate pathway such as dihydrodipicolinate synthase gene (dapA), aspartokinase gene (lysC), dihydrodipicolinate reductase gene (dapB), diaminopimelate decarboxylase gene (lysA), diaminopimelate dehydrogenase gene (ddh) (WO96/40934 for all the foregoing genes), phosphoenolpyrvate carboxylase gene (ppc) (Japanese Patent Laid-open No. 60-87788), aspartate aminotransferase gene (aspC) (Japanese Patent Publication No. 6-102028), diaminopimelate epimerase gene (dapF) (Japanese Patent Laid-open No. 2003-135066), and aspartate semialdehyde dehydrogenease gene (asd) (WO00/61723), genes encoding enzymes of the aminoadipic acid pathway such as homoaconitate hydratase gene (Japanese Patent Laid-open No. 2000-157276), and so forth. Coryneform bacteria modified by using these genes are disclosed in Japanese Patent Laid-open Nos. 10-215883, 10-165180, WO96/40934, etc.

The gene encoding aspartokinase III (lysC can be modified so that the enzyme is desensitized to feedback inhibition by L-lysine. Such a modified lysC gene for desensitization to the feedback inhibition can be obtained by the method described in U.S. Pat. No. 5,932,453.

Furthermore, coryneform bacteria imparted with L-lysine-producing ability may have reduced activity of an enzyme that catalyzes a reaction producing a compound other than L-lysine or may be deficient in such an activity, or they may have reduced activity of an enzyme that negatively acts on L-lysine production, or may be deficient in such an activity. Examples of such enzymes include homoserine dehydrogenase, lysine decarboxylase (cadA, ldcC), and malic enzyme, and strains in which activities of these enzymes are decreased or deleted are disclosed in WO95/23864, and so forth.

Examples of coryneform bacteria imparted with L-tryptophan-producing ability are bacteria in which one or two or more activities among the anthranilate synthetase activity, phosphoglycerate dehydrogenase activity, and tryptophan synthase activity are enhanced. Since anthranilate synthetase and phosphoglycerate dehydrogenase suffer from feedback inhibition by L-tryptophan and L-serine, respectively, their enzymatic activities can be enhanced by mutating the enzyme so that it is desensitized to these L-amino acids.

Furthermore, L-tryptophan-producing ability can also be imparted by introducing a recombinant DNA containing the tryptophan operon. Moreover, L-tryptophan-producing ability may be improved or imparted by enhancing expression of a gene encoding tryptophan synthase in the tryptophan operon (trpBA). Tryptophan synthase includes α and β subunits, which are encoded by the trpA and trpB genes, respectively. The nucleotide sequence of the tryptophan operon and the nucleotide sequences of trpA and trpB are registered as GenBank Accession No. J01714 (WO2005/103275).

Examples of coryneform bacteria imparted with L-tryptophan-producing ability include Brevibacterium flavum AJ11667 (refer to Japanese Patent Laid-open No. 57-174096).

Examples of coryneform bacteria imparted with L-tyrosine-producing ability include Corynebacterium glutamicum AJ11655 (FERM P-5836, refer to Japanese Patent Publication No. 2-6517), and Brevibacterium lactofermentum AJ12081 (FERM P-7249, refer to Japanese Patent Laid-open No. 60-70093).

Examples of coryneform bacteria having L-phenylalanine-producing ability include the strain showing tyrosine auxotrophy and L-phenylalanyl-L-tyrosine resistance (Japanese Patent Laid-open No. 5-49489) and Brevibacterium lactofermentum AJ12637 (FERM BP-4160, refer to the French Patent Laid-open No. 2,686,898).

L-Tryptophan, L-phenylalanine, and L-tyrosine are all aromatic amino acids and share a common biosynthesis pathway. Examples of the genes encoding the biosynthetic enzymes for these aromatic amino acids include deoxyarabino-heptulosonate phosphate synthase (aroG), 3-dehydroquinate synthase (aroB), shikimic acid dehydratase, shikimate kinase (aroL), 5-enolpyruvylshikimate-3-phosphate synthase (aroA), and chorismate synthase (aroC) (European Patent Laid-open No. 763127). Therefore, an ability to produce an aromatic amino acid can be improved by increasing the copy number of a gene encoding any of these enzymes on a plasmid or genome. It is known that these genes are controlled by the tyrosine repressor (tyrR), and so activity of an aromatic amino acid biosynthetic enzyme may also be increased by deleting the tyrR gene (see European Patent Laid-open No. 763127).

Furthermore, examples of coryneform bacteria having L-threonine-producing ability include Corynebacterium acetoacidophilum AJ12318 (FERM BP-1172, refer to U.S. Pat. No. 5,188,949), and so forth.

Examples of coryneform bacteria imparted with L-leucine-producing ability include Brevibacterium lactofermentum AJ3718 (FERM P-2516, 2-thiazolealanine and β-hydroxyleucine resistant, and isoleucine and methionine auxotrophic).

Examples of coryneform bacteria having L-isoleucine-producing ability include Brevibacterium flavum AJ12149 (FERM BP-759, refer to U.S. Pat. No. 4,656,135), and so forth.

<1-2> Enhancement of Carbonic Anhydrase Activity

Coryneform bacteria imparted with an L-amino acid-producing ability as mentioned above can be modified so that the carbonic anhydrase activity is enhanced. However, either the modification for enhancing the carbonic anhydrase activity or the impartation of an L-amino acid-producing ability can be performed first.

The expression “has been modified to enhance carbonic anhydrase activity” can include a state where the number of carbonic anhydrase molecules per cell has been increased, as well as a state where the activity per molecule of carbonic anhydrase has been increased, compared with a parent or wild-type strain, or the like. Furthermore, the wild-type strain used as the object of the comparison may be, for example, the Corynebacterium glutamicum (Brevibacterium lactofermentum) ATCC 13869 strain or ATCC 13032 strain.

The enhancement of the carbonic anhydrase activity can be confirmed by comparing the carbonic anhydrase activity or amount of mRNA of a gene encoding the carbonic anhydrase with that of a wild-type or unmodified strain. Examples of method for confirming expression amount include Northern hybridization and RT-PCR (Molecular Cloning, Cold Spring Harbor Laboratory Press, Cold Spring Harbor, USA, 2001). The enzyme activity or expression amount can be increased to any level so long as it is increased compared with that of a wild-type or unmodified strain, and for example, it can be increased not less than 1.5 times, not less than 2 times, or even not less than 3 times, as compared with that of, for example, a wild-type or an unmodified strain.

Carbonic anhydrase is an enzyme involved in the mutual conversion of carbon dioxide and bicarbonate radical (EC 4.2.1.1). The carbonic anhydrase activity can be measured by the method of Wilbur et al, (Wilbur K. M., Anderson N. G, Electrometric and Colorimetric Determination of Carbonic Anhydrase, J. Biol. Chem., 176:147-154, 1948).

As genes encoding carbonic anhydrase (ca gene) of coryneform bacteria, two kinds of genes, the gene encoding β type carbonic anhydrase (bca) and gene encoding γ type carbonic anhydrase (gca), have been reported. NCg12579 of the C. glutamicum ATCC 13032 strain registered at GenBank (corresponding to bca, complementary strand of 2837954 to 2838577 of Accession BA_{—}000036.3) can be used. The nucleotide sequence of the gene is shown as SEQ ID NO: 13 (coding region corresponds to the nucleotide numbers 1 to 621), and the amino acid sequence of the encoded protein is shown as SEQ ID NO: 14. Furthermore, the nucleotide sequence of the bca gene of the C. glutamicum ATCC 13869 strain is shown as the nucleotide numbers 562 to 1182 in SEQ ID NO: 11, and the amino acid sequence of the encoded protein is shown as SEQ ID NO: 12.

Furthermore, as the gene, a homologue gene of the ca gene derived from another microorganism may be used, so long as it can express a protein that shows the carbonic anhydrase activity in coryneform bacteria. Such a homologue of the ca gene can be searched for by using BLAST or the like with reference to the nucleotide sequence of the nucleotide numbers 562 to 1182 of SEQ ID NO: 11 or the nucleotide sequence of SEQ ID NO: 13 (blast.genome.jp/).

Since the sequence of the bca gene has already been elucidated, a region including bca and a control region of bca can be obtained by PCR using primers produced on the basis of the above nucleotide sequence, for example, the primers shown as SEQ ID NOS: 7 and 8, and a chromosomal DNA of a coryneform bacterium as a template. Homologues of bca of other microorganisms can also be obtained in a similar manner.

Furthermore, since the nucleotide sequence of the bca gene can differ depending on the species or strain of coryneform bacteria, the bca gene is not limited to the nucleotide sequence of nucleotide numbers 562 to 1182 of SEQ ID NO: 11 or the nucleotide sequence of SEQ ID NO: 13, but it can be a mutant or artificially modified gene that codes for a protein having the sequence of SEQ ID NO: 12 or 14, but which includes substitutions, deletions, insertions, additions, etc. of one or several amino acid residues at one or more positions so long as the protein has the carbonic anhydrase activity. Although the number meant by the term “one or several” can differ depending on positions in the three-dimensional structure of the protein or types of amino acid residues, specifically, it can be 1 to 20, 1 to 10, or even 1 to 5. The substitutions, deletions, insertions, additions, inversions or the like of amino acid residues described above can also include those caused by a naturally occurring mutation based on individual differences, or differences in species of microorganisms that contain the bca gene (mutant or variant).

The aforementioned substitution can be a conservative substitution that is a neutral substitution, that is, one that does not result in a functional change. The conservative mutation can be a mutation wherein substitution takes place mutually among Phe, Trp and Tyr, if the substitution site is an aromatic amino acid; among Leu, Ile and Val, if the substitution site is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having hydroxyl group. Specific examples of conservative substitutions include: substitution of Ser or Thr for Ala; substitution of Gln, His or Lys for Arg; substitution of Glu, Gln, Lys, His or Asp for Asn; substitution of Asn, Glu or Gln for Asp; substitution of Ser or Ala for Cys; substitution of Asn, Glu, Lys, His, Asp or Arg for Gln; substitution of Gly, Asn, Gln, Lys or Asp for Glu; substitution of Pro for Gly; substitution of Asn, Lys, Gln, Arg or Tyr for His; substitution of Leu, Met, Val or Phe for Ile; substitution of Ile, Met, Val or Phe for Leu; substitution of Asn, Glu, Gln, His or Arg for Lys; substitution of Ile, Leu, Val or Phe for Met; substitution of Trp, Tyr, Met, Ile or Leu for Phe; substitution of Thr or Ala for Ser; substitution of Ser or Ala for Thr; substitution of Phe or Tyr for Trp; substitution of His, Phe or Trp for Tyr; and substitution of Met, Ile or Leu for Val.

Furthermore, the bca gene can include a nucleotide sequence encoding a protein having an identity not less than 80%, less than 90%, not less than 95%, or even not less than 97%, to the entire amino acid sequence of SEQ ID NO: 12 or 14, and having the carbonic anhydrase activity. Furthermore, the degree of degeneracy of the gene can vary depending on the host into which the bca gene is introduced, and therefore codons can be replaced with those which are favorable for the chosen host.

Moreover, the bca gene can code for a protein with an elongated or deleted N- or C-terminal sequence, so long as the protein has the carbonic anhydrase activity. The length of the amino acid sequence to be elongated or deleted can be 50 amino acid residues or less, 20 or less, 10 or less, or even 5 or less. More specifically, the bca gene can encode a protein having the amino acid sequence of SEQ ID NO: 12 or 24, but wherein the sequence is elongated by 5 to 50 amino acid residues on the N-terminal or C-terminal side, or 5 to 50 residues are deleted on either side.

Such genes homologous to the bca gene described above can be obtained by modifying the nucleotide sequence of nucleotide numbers 562 to 1182 of SEQ ID NO: 11 or the nucleotide sequence of SEQ ID NO: 13 so that the protein encoded by the gene includes substitutions, deletions, insertions, or additions of amino acid residues at a specific site(s), for example, by site-specific mutagenesis. Furthermore, homologous genes can also be obtained by conventionally known mutation treatments, such as those described below. For example, the nucleotide sequence mentioned above can be treated with hydroxylamine or the like in vitro, or the microorganism, for example, coryneform bacteria, containing the gene can be treated with ultraviolet ray irradiation or a mutagen typically used for mutation, such as N-methyl-N′-nitro-N-nitrosoguanidine (NTG) or ethyl methanesulfonate (EMS), or a mutation can be artificially introduced into bca by genetic recombination based on error-prone PCR, DNA shuffling, or StEP-PCR, so that a highly active bca gene can be obtained (Firth A. E., Patrick W. M., Bioinformatics, 2005 Jun. 2, Statistics of Protein Library Construction).

Examples of the bca gene also include a DNA that hybridizes with a complement of the nucleotide sequence of nucleotide numbers 562 to 1182 of SEQ ID NO: 11 or the nucleotide sequence of SEQ ID NO: 13, or a probe that can be prepared from these sequences under stringent conditions and codes for a protein which has the carbonic anhydrase activity. The “stringent conditions” can be conditions under which a so-called specific hybrid is formed, and non-specific hybrid is not formed. Examples include, for example, conditions under which DNAs having high homology to each other, for example, DNAs having a homology of, for example, not less than 80%, not less than 90%, not less than 95%, or not less than 97%, hybridize with each other, and DNAs having homology lower than the above levels do not hybridize with each other. “Stringent conditions” can also include washing conditions which are typical in Southern hybridization, for example, washing once, or twice or three times, at salt concentrations and a temperature of 1×SSC, 0.1% SDS at 60° C., 0.1×SSC, 0.1% SDS at 60° C., or even 0.1×SSC, 0.1% SDS at 68° C.

A partial sequence of the nucleotide sequence of the nucleotide numbers 562 to 1182 of SEQ ID NO: 11 or the nucleotide sequence of SEQ ID NO: 13 can also be used as the probe. Such a probe can be prepared by PCR using oligonucleotides prepared on the basis of either of these nucleotide sequences as primers and a DNA fragment containing any one of the nucleotide sequence of the nucleotide numbers 562 to 1182 of SEQ ID NO: 11 and the nucleotide sequence of SEQ ID NO: 13 as a template. When a DNA fragment having a length of about 300 bp is used as the probe, for example, the washing conditions after hybridization under the aforementioned conditions can be exemplified by 2×SSC, 0.1% SDS at 50° C.

Expression of the bca gene can be enhanced by increasing the copy number of the bca gene. For example, the copy number of the gene can be increased by ligating a fragment containing the bca gene to a vector, such as a multi copy vector, that functions in coryneform bacteria, to prepare a recombinant DNA, and transforming such a microorganism having an L-amino acid-producing ability as mentioned above with the DNA. Alternatively, after the transformation of a wild-type strain of a coryneform bacterium by introducing such a recombinant DNA as mentioned above, the ability to produce an L-amino acid can be imparted to the transformed bacterium. The copy number of the gene can also be increased by transferring a single copy or multiple copies of the bca gene to the bacterial chromosome. Transfer of the bca gene to the chromosome can be confirmed by Southern hybridization using a portion of the bca gene as a probe.

Expression of the bca gene can also be enhanced by modifying an expression control sequence of the bca gene. For example, the promoter sequence of the bca gene can be replaced with a stronger promoter, or by making a promoter sequence closer to a consensus sequence (WO00/18935).

Methods for modifying a coryneform bacterium to enhance carbonic anhydrase activity are explained below. These methods can be performed as described in a manual such as Molecular Cloning (Cold Spring Harbor Laboratory Press, Cold Spring Harbor (USA), 2001).

Expression of the bca gene can be increased by increasing the copy number of the bca gene, and the copy number can be increased by amplifying the bca gene using a plasmid as described below. First, the bca gene is cloned from chromosome of a coryneform bacterium. Chromosomal DNA can be prepared from a bacterium as a DNA donor, for example, by the method of Saito and Miura (see H. Saito and K. Miura, Biochem. Biophys. Acta, 72, 619, 1963; Text for Bioengineering Experiments, Edited by the Society for Bioscience and Bioengineering, Japan, 97-98, Baifukan Co., Ltd., 1992), or the like. Oligonucleotides for use in PCR can be synthesized on the basis of the aforementioned known information, and for example, the synthetic oligonucleotides shown in SEQ ID NOS: 7 and 8 can be used to amplify the bca gene.

A gene fragment including the bca gene amplified by PCR can be ligated to a vector DNA autonomously replicable in cells of Escherichia coli and/or coryneform bacteria to prepare a recombinant DNA, and this recombinant DNA can be introduced into Escherichia coli, which makes the operation thereafter easier. Examples of vectors autonomously replicable in a cell of Escherichia coli include pUC19, pUC18, pHSG299, pHSG399, pHSG398, RSF1010, pBR322, pACYC184, pMW219, and so forth.

The aforementioned DNA is introduced into a vector that functions in coryneform bacteria. The vector that functions in coryneform bacteria is, for example, a plasmid autonomously replicable in coryneform bacteria. Specific examples of the plasmid that is autonomously replicable in coryneform bacteria include plasmid pCRY30 described in Japanese Patent Laid-open No. 3-210184; plasmids pCRY21, pCRY2KE, pCRY2KX, pCRY31, pCRY3KE, and pCRY3KX described in Japanese Patent Laid-open No. 2-72876 and U.S. Patent No. 5,185,262; plasmids pCRY2 and pCRY3 described in Japanese Patent Laid-open No. 1-191686; pAM330 described in Japanese Patent Laid-open No. 58-67679; pHM1519 described in Japanese Patent Laid-open No. 58-77895; pAJ655, pAJ611, and pAJ1844 described in Japanese Patent Laid-open No. 58-192900; pCG1 described in Japanese Patent Laid-open No. 57-134500; pCG2 described in Japanese Patent Laid-open No. 58-35197; pCG4, pCG11 etc. described in Japanese Patent Laid-open No. 57-183799; and pVK7 described in Japanese Patent Laid-open No. 10-215883.

Furthermore, if a DNA fragment which enables a plasmid to autonomously replicate in coryneform bacteria is excised from any of those vectors and the fragment is inserted into any of the aforementioned vectors for Escherichia coli, the resulting vector can be used as a so-called shuttle vector which is autonomously replicable both in Escherichia coli and coryneform bacteria.

To prepare a recombinant DNA by ligation of the bca gene to a vector that functions in coryneform bacteria, the vector is digested with a restriction enzyme suitable for the ends of the gene. Such a restriction enzyme site may be introduced in advance into a synthetic oligonucleotide to be used for amplifying the bca gene. Ligation can be performed by using a ligase such as T4 DNA ligase.

In order to introduce a recombinant plasmid prepared as described above into a coryneform bacterium, any known transformation method can be employed. For example, recipient cells can be treated with calcium chloride so as to increase permeability for the DNA, which has been reported for Escherichia coli K-12 (Mandel, M. and Higa, A., J. Mol. Biol., 53, 159, 1970). Also, competent cells can be prepared from growing cells and DNA can be introduced into these cells, which has been reported for Bacillus subtilis (Duncan, C. H., Wilson, G. A. and Young, F. E., Gene, 1, 153, 1977). Another method is to make DNA recipient cells into protoplasts or spheroplasts which easily take up a recombinant DNA, and a recombinant DNA can be introduced into these cells, which are known for Bacillus subtilis, actinomycetes, and yeasts (Chang, S. and Choen, S. N., Mol. Gen. Genet., 168, 111, 1979; Bibb, M. J., Ward, J. M. and Hopwood, O. A., Nature, 274, 398, 1978; Hinnen, A., Hicks, J. B. and Fink, G. R., Proc. Natl. Acad. Sci. USA, 75, 1929, 1978). In addition, coryneform bacteria can also be transformed by the electric pulse method (Japanese Patent Laid-open No. 2-207791) or by the conjugal transfer method (Biotechnology (NY). 1991 January; 9(1):84-7).

The copy number of the bca gene can also be increased by integrating multiple copies of the bca gene into the chromosomal DNA of the coryneform bacterium, which can be accomplished by homologous recombination. This technique is performed by targeting a sequence which is present in multiple copies on the chromosomal DNA. Such sequences can include a repetitive DNA or inverted repeats present at the end of a transposable element. Alternatively, as disclosed in Japanese Patent Laid-open No. 2-109985, multiple copies of the bca gene can be introduced into a chromosomal DNA by incorporating them into a transposon, and transferring the transposon (Japanese Patent Laid-open Nos. 2-109985, 7-107976; Mol. Gen. Genet., 245, 397-405, 1994; Plasmid, 2000 November; 44(3): 285-91).

Also conceivable is a method of inserting the bca gene into a plasmid having a replication origin that cannot be replicated in a host, or a plasmid having a replication origin that cannot replicate in a host and an ability to cause conjugal transfer to a host, and introducing the obtained plasmid into the host to amplify the gene on a chromosome. Examples of such a plasmid include pSUP301 (Simo et al., Bio/Technology 1, 784-791, 1983), pK18mob or pK19mob (Schaefer et al., Gene, 145, 69-73, 1994), pGEM-T (Promega Corporation, Madison, Wis., USA), pCR2.1-TOPO (Shuman, Journal of Biological Chemistry, 269:32678-84, 1994; U.S. Pat. No. 5,487,993), pCR Blunt (Invitrogen, Groningen, Netherlands; Bernard et al., Journal of Molecular Biology, 234: 534-541, 1993), pEM1 (Schrumpf et al., Journal of Bacteriology, 173:4510-4516, 1991), pBGS8 (Spratt et al., Gene, 41:337-342, 1986), and so forth. A plasmid vector which includes the bca gene can be transferred into a coryneform bacterium by conjugation or transformation to transfer the gene into a chromosome. The conjugation method is described by, for example, Schaefer et al. (Applied and Environmental Microbiology, 60, 756-759, 1994). The transformation method is described by, for example, Theirbach et al. (Applied Microbiology and Biotechnology, 29, 356-362, 1988), Dunican and Shivinan (Bio/Technology 7, 1067-1070, 1989), and Tauch et al. (FEMS Microbiological Letters, 123, 343-347, 1994).

The activity of Bca can also be enhanced by replacing a native expression control sequence, such as a promoter, of the bca gene, on the chromosomal DNA or a plasmid with a stronger one. Other methods include modifying a factor involved in expression control of the bca gene, such as operator or repressor, or ligating a strong terminator (Hamilton et al., Journal of Bacteriology 171:4617-4622). For example, the lac promoter, trp promoter, trc promoter, PS2 promoter, and so forth are known as strong promoters. Methods for evaluating the strength of promoters and examples of strong promoters are described in the paper of Goldstein et al. (Prokaryotic Promoters in Biotechnology, Biotechnol. Annu. Rev., 1, 105-128, 1995), and so forth. Furthermore, as disclosed in WO00/18935, strength of a promoter can be increased by making several nucleotide substitutions in the promoter region of a target gene so as to make the sequence closer to a consensus sequence. For example, the −35 region can be replaced with TTGACA or TTGCCA, and the −10 region can be replaced with TATAAT or TATAAC. In addition, it is known that the translation efficiency of mRNA is significantly affected by substituting several nucleotides in the spacer region between the ribosome-binding site (RBS) and the translation initiation codon, in particular, the sequence immediately upstream of the initiation codon, and they can be modified.

Examples of the upstream region of the bca gene include, for example, the region of the nucleotide numbers 1 to 561 of SEQ ID NO: 11. An expression control sequence such as promoter upstream of the bca gene may also be identified by using a promoter search vector or gene analysis software such as GENETYX. By such substitution or modification of the promoter as described above, expression of the bca gene can be enhanced. Substitution of an expression control sequence can be attained by, for example, using a temperature sensitive plasmid. Modification of an expression control sequence can be combined with increasing of copy number of the bca gene.

Increasing the expression amount can also be attained by extending the survival time of the mRNA or by preventing degradation of the encoded protein in the cells.

Genes encoding carbonic anhydrase of Escherichia coli have been reported, and include the yadF and cynT genes, which encode two kinds of carbonic anhydrases, Can (carbonic anhydrase 2) and CynT (carbonic anhydrase 1), respectively., These can also be used instead of the aforementioned bca gene. As yadF, yadF of Escherichia coli registered at GenBank (Accession EG_{—}12319) is exemplified. The nucleotide sequence of this gene is shown as SEQ ID NO: 27 (coding region: 201 to 860), and the encoded amino acid sequence is shown as SEQ ID NO: 28. As the cynT gene, cynT of Escherichia coli (Accession EG_{—}10176) is exemplified. The nucleotide sequence of this cynT gene is shown as SEQ ID NO: 29 (coding region: 201 to 857), and the encoded amino acid sequence is shown as SEQ ID NO: 30. A gene encoding a protein showing 80% or more, 90% or more, 95% or more, or even 97% or more, of identity for the full length of any of these amino acid sequences, and having the carbonic anhydrase activity can also be used.

The microorganism chosen for the production method as described herein can be a microorganism modified to impart D-xylose-5-phosphate phosphoketolase activity and/or fructose-6-phosphate phosphoketolase activity in addition to enhancing the carbonic anhydrase activity.

Either one or both of the activities of D-xylose-5-phosphate phosphoketolase and fructose-6-phosphate phosphoketolase can be imparted. In this specification, D-xylose-5-phosphate phosphoketolase and fructose-6-phosphate phosphoketolase can be collectively referred to as phosphoketolase.

The D-xylose-5-phosphate phosphoketolase activity can mean an activity of converting xylose-5-phosphate into glycelaldehyde-3-phosphate and acetyl phosphate by consuming phosphoric acid to release one molecule of H₂O. This activity can be measured by the method described by Goldberg, M. et al. (Methods Enzymol., 9, 515-520, 1996) or by the method described by L. Meile (J. Bacteriol., 183:2929-2936, 2001).

The fructose-6-phosphate phosphoketolase activity can mean an activity of converting fructose-6-phosphate into erythrose-4-phosphate and acetyl phosphate by consuming phosphoric acid to release one molecule of H₂O. This activity can be determined by the method described by Racker, E. (Methods Enzymol., 5, 276-280, 1962) or by the method described by L. Meile (J. Bacteriol., 183:2929-2936, 2001).

The phosphoketolase activity can be imparted by introducing a gene encoding a phosphoketolase into the cells of a coryneform bacterium by using a plasmid, by incorporating such a gene into the chromosome of a coryneform bacterium, or the like.

Coryneform bacteria do not inherently have the phosphoketolase activity, but the phosphoketolase activity can be imparted by introducing a plasmid containing a gene encoding a phosphoketolase derived from another organism into the cells of a coryneform bacterium, by incorporating a gene encoding a phosphoketolase derived from another organism into chromosome of a coryneform bacterium, or the like.

A gene encoding D-xylose-5-phosphate phosphoketolase can be obtained by PCR using chromosomal DNA of a microorganism having the D-xylose-5-phosphate phosphoketolase activity as a template, or the like. Examples of such a microorganism include bacteria such as lactic acid bacteria, methanol-assimilating bacteria, methane-assimilating bacteria, bacteria belonging to the genus Streptococcus, Acetobacter, Bifidobacterium, Lactobacillus, Thiobacillus, Methylococcus, Butyrivibrio, Fibrobactor or the like; yeasts belong to the genus Candida, Rhodotorula, Rhodosporidium, Pichia, Yarrowia, Hansenula, Kluyveromyces, Saccharomyces, Trichosporon, Wingea, or the like, etc.

A gene encoding fructose-6-phosphate phosphoketolase can be obtained by PCR using chromosomal DNA of a microorganism having the fructose-6-phosphate phosphoketolase activity as a template, or the like. Examples of such a microorganism include bacteria belonging to the genus Acetobacter, Bifidobacterium, Chlorobium, Brucella, Methylococcus, Gardnerella, or the like; yeasts belong to the genus Candida, Rhodotorula, Saccharomyces, or the like, etc.

A specific example of the gene encoding D-xylose-5-phosphate phosphoketolase is the xpkA gene encoding D-xylose-5-phosphate phosphoketolase of Lactobacillus pentosus MD363. The nucleotide sequence thereof is registered at the EMBL/GenBank database with an accession number of AJ309011 (Posthuma, C C. et al, Appl. Environ. Microbiol., 68(2), 831-7, 2002, SEQ ID NO: 15).

The xpk1 gene derived from Lactobacillus plantarum can also be used. The nucleotide sequence thereof is registered at the EMBL/GenBank database with an accession number of NC_{—}004567 Region: complement (2362936 to 2365302) (Kleerebezem, M., et al, Proc. Natl. Acad. Sci. U.S.A. 100 (4), 1990-1995, 2003, SEQ ID NO: 17).

In addition, examples of homologues of these genes include a gene of Lactobacillus plantarum as GenBank Accession No. NC_{—}004567 complement (3169067 to 3171478), a gene of Streptococcus agalactiae encoding the amino acid sequence of GenBank Accession No. NP_{—}736274, a gene of Lactococcus lactis subsp. Lactis encoding the amino acid sequence of GenBank Accession No. NP_{—}267658, a gene of Lactobacillus johnsonii which is registered as GenBank Accession No. NC_{—}005362 (696462 to 698867), a gene of Lactobacillus acidophilus encoding the amino acid sequence of GenBank Accession No. YP_{—}193510, and so forth.

A gene encoding a protein having the activities of both D-xylose-5-phosphate phosphoketolase and fructose-6-phosphate phosphoketolase can also be used. Examples of such a gene include the xfp gene of Bifidobacterium lactis. The nucleotide sequence thereof is registered at the EMBL/GenBank database as accession number of AJ293946 (Meile, L. et al, J. Bacteriol., 183(9), 2929-36, 2001, SEQ ID NO: 19).

Homologues of the xfp gene include a gene of Bifidobacterium longum encoding the amino acid sequence of GenBank Accession No. NP_{—}696135, a gene of Chlorobium tepidum encoding the amino acid sequence of GenBank Accession No. NP_{—}662409, a gene of Brucella suis encoding the amino acid sequence of GenBank Accession No. NP_{—}699578, a gene of Brucella abortus encoding the amino acid sequence of GenBank Accession No. YP_{—}223570, and so forth.

In addition, the phosphoketolase gene may be a DNA hybridizable with a complement of any of the aforementioned nucleotide sequences or a probe that can be prepared from the complement under stringent conditions, and encoding a protein having the phosphoketolase activity. Furthermore, there may be also used a DNA encoding a protein showing 80% or more, 90% or more, 95% or more, or even 97% or more, of identity to the full length of the amino acid sequence of SEQ ID NO: 16, 18, or 20, and having the phosphoketolase activity.

The microorganism used for the production method can be a microorganism modified to enhance phosphotransacetylase activity as compared with a wild-type strain, in addition to the enhancement of the carbonic anhydrase activity. The phosphotransacetylase is an enzyme involved in the acetic acid metabolism. In Escherichia coli, it is responsible for the reaction that generates acetyl phosphate from phosphoric acid and acetyl-CoA, which is a part of the main pathway of the acetic acid generation. It is known that, on the other hand, in Corynebacterium glutamicum, the activity of phosphotransacetylase increases when acetic acid is assimilated, and acetyl-CoA is generated. Moreover, it is also known that RamB, which is a transcription factor, is involved in the negative control of the phosphotransacetylase activity (Microbiology, 145, 503-513, 1999; Journal of Bacteriology, 186, 9, 2798-2809, 2004). The phosphotransacetylase activity can be enhanced by increasing the copy number of a gene encoding phosphotransacetylase, modifying a promoter of a gene encoding phosphotransacetylase, or the like, as in the case of the enhancement of the phosphotransacetylase activity mentioned above. Furthermore, the enhancement may also be attained by deleting the ramB gene mentioned above, or modifying the RamB protein-binding site located upstream of the gene encoding phosphotransacetylase.

As the gene encoding phosphotransacetylase (pta gene) of a coryneform bacterium, the nucleotide sequence NCg12657 of ATCC 13032 registered at Genbank (complementary strand of 2936506 to 2937891 of Accession NC 003450.3) can be used. The nucleotide sequence of this gene is shown as SEQ ID NO: 21, and the encoded amino acid sequence is shown as SEQ ID NO: 22, respectively. Furthermore, the nucleotide sequence of the pta gene of the C. glutamicum ATCC 13869 strain is shown as the nucleotide numbers 1214 to 2641 in SEQ ID NO: 23, and the amino acid sequence encoded by the gene is shown as SEQ ID NO: 24.

Furthermore, a homologue gene of the pta gene derived from another microorganism may be used so long as it encodes a protein having the phosphotransacetylase activity in coryneform bacteria. Such a homologue of the pta gene can be searched for by using BLAST (blast.genome.jp/) or the like with reference to the nucleotide sequence of the nucleotides numbers 1214 to 2641 of SEQ ID NO: 23.

The nucleotide sequence of the pta gene has already been elucidated. Therefore, a region containing the pta gene and a expression control sequence thereof can be obtained by PCR (polymerase chain reaction, refer to White, T. J. et al., Trends Genet. 5, 185, 1989) using primers prepared on the basis of the known nucleotide sequence, for example, the primers of SEQ ID NOS: 25 and 26, and chromosomal DNA of a coryneform bacterium as a template. A homologue of the pta gene derived from another microorganism can also be obtained in the same manner.

The nucleotide sequence of the pta gene can differ depending on the species or strain of coryneform bacteria, the pta gene is not limited to the nucleotide sequence of the nucleotide numbers 1214 to 2641 of SEQ ID NO: 23 or the nucleotide sequence of SEQ ID NO: 21, and it can be a mutant or artificially modified gene that codes for a protein having the sequence of SEQ ID NO: 24 or 22, but which includes substitutions, deletions, insertions, additions, etc. of one or several amino acid residues at one or more positions so long as the encoded protein has the function of the Pta protein, the phosphotransacetylase activity. Although the number meant by the term “one or several” can differ depending on positions in the three-dimensional structure of the protein or types of amino acid residues, specifically, it can be 1 to 20, 1 to 10, or even 1 to 5. The substitutions, deletions, insertions, additions, inversions or the like of amino acid residues described above can also include those caused by a naturally occurring mutation based on individual differences, differences in species of microorganisms that contain the pta gene (mutant or variant), or the like.

Furthermore, a DNA encoding a protein showing an identity not less than 80%, not less than 90%, not less than 95%, or even not less than 97%, to the entire amino acid sequence of SEQ ID NO: 24 or 22, and having the phosphotransacetylase activity can also be used.

The coryneform bacterium may be a microorganism modified to enhance pyruvate carboxylase activity as compared with a wild-type strain, in addition to the aforementioned modifications. As the pyruvate carboxylase gene, for example, genes derived from coryneform bacteria and Bacillus bacteria can be used, and the pyc gene of the C. glutamicum ATCC 13032 strain (GenBank Accession No. NCg10659) and the pyc gene of B. subtilis (European Patent No. 1092776) can be used.

The coryneform bacterium can be a bacterium modified to enhance phosphoenolpyruvate carboxylase activity as compared with a wild-type strain, in addition to the aforementioned modifications. As the phosphoenolpyruvate carboxylase gene, for example, genes derived from coryneform bacteria and Escherichia bacteria can be used, and the ppc gene of the C. glutamicum ATCC 13032 strain (GenBank Accession No. NCgll523) and the ppc gene derived from the E. coli MG1655 strain (GenBank Accession No. NP_{—}418391) can be used.

Since the phosphoenolpyruvate carboxylase may suffer from feedback inhibition by aspartic acid, it can be modified so that it is desensitized to the feedback inhibition by aspartic acid (European Patent No. 0723011).

<2> Method for Producing L-amino Acid

An L-amino acid can be produced by culturing a coryneform bacterium obtained as described above in a medium to produce and accumulate the L-amino acid in the medium, and collecting the L-amino acid from the medium.

As the medium used for the culture, a typical medium containing a carbon source, nitrogen source, and mineral salts as well as organic trace nutrients such as amino acids and vitamins as required can be used. Either a synthetic or a natural medium can be used. Any kind of carbon source and nitrogen source can be used for the medium so long as they can be utilized by the chosen strain to be cultured.

As the carbon source, sugars such as glucose, glycerol, fructose, sucrose, maltose, mannose, galactose, starch hydrolysates and molasses can be used. In addition, organic acids such as acetic acid and citric acid, and alcohols such as ethanol can also be used each alone or in combination with other carbon sources. As the nitrogen source, ammonia, ammonium salts such as ammonium sulfate, ammonium carbonate, ammonium chloride, ammonium phosphate and ammonium acetate, nitric acid salts and so forth can be used. As the organic trace nutrients, amino acids, vitamins, fatty acids, nucleic acids, those containing those substances such as peptone, casamino acid, yeast extract, and soybean protein decomposition product, and so forth can be used. When an auxotrophic mutant strain that requires an amino acid or the like for its growth is used, the required nutrient is preferably supplemented. As the inorganic salts, phosphoric acid salts, magnesium salts, calcium salts, iron salts, manganese salts and so forth can be used.

The culture can be performed as an aerobic culture, while the fermentation temperature can be controlled to be 20 to 45° C., and pH to be 3 to 9. When the pH decreases during the culture, calcium carbonate may be added, or culture is neutralized with an alkaline substance such as ammonia gas. The target L-amino acid such as L-glutamic acid is accumulated in a marked amount in the culture medium after, for example, 10 to 120 hours of the culture under such conditions as described above.

Moreover, when L-glutamic acid is produced, the culture can be performed by precipitating L-glutamic acid in a medium by using, as the medium, a liquid medium adjusted to satisfy a condition under which L-glutamic acid is precipitated. Examples of the conditions under which L-glutamic acid is precipitated include, for example, pH of 5.0 to 4.0, pH 4.5 to 4.0, pH 4.3 to 4.0, or even pH 4.0 (European Patent Laid-open No. 1078989).

The L-amino acid can be collected from the culture medium after the culture by a known collection method. For example, after the cells are removed from the culture medium, the L-amino acid can be collected by concentrating the medium to crystallize the L-amino acid, ion exchange chromatography, or the like. When the culture is performed under such conditions that L-glutamic acid is precipitated, L-glutamic acid which precipitates in the medium can be collected by centrifugation or filtration. In this case, L-glutamic acid which dissolves in the medium may be crystallized and then separated together with already precipitated L-glutamic acid.

### EXAMPLE

Hereafter, the present invention will be specifically explained with reference to the following non-limiting example.

1. Construction of bca-Amplified C. glutamicum ATCC 13869 Strain

A sucA-deficient strain was used as a parent strain for bca gene amplification. A sucA-deficient strain can be constructed by the method described below.

(1-1) Construction of sucA-Deficient Strain

A sucA-deficient strain of ATCC 13869 (ATCC13869ΔsucA) was constructed as follows.

The sucA gene encoding the E1o subunit of α-ketoglutarate dehydrogenase was disrupted by using the plasmid pBS3 carrying the sacB gene encoding levan sucrase. For construction of a sacB-carrying vector for gene disruption, pBS3 described in International Patent Publications WO2005/113745 and WO2005/113744 was used.

A gene fragment that includes sucA derived from the C. glutamicum ATCC 13869 strain, but lacking the ORF, was obtained by overlap PCR using, as primers, synthetic DNAs designed with reference to the nucleotide sequence of the gene of C. glutamicum ATCC 13032 which has already been reported (SEQ ID NO: 9, GenBank Database Accession No.NC_{—}003450). Specifically, PCR was performed in a conventional manner with the chromosomal DNA of the C. glutamicum ATCC 13869 strain as a template and the synthetic DNAs of SEQ ID NOS: 1 and 2 as primers to obtain an amplification product of the N-terminus side of the sucA gene. Separately, in order to obtain an amplification product of the C-terminus side of the sucA gene, PCR was performed in a conventional manner with the genomic DNA of the C. glutamicum ATCC 13869 strain as a template and the synthetic DNAs of SEQ ID NOS: 3 and 4 as primers. The nucleotide sequences of SEQ ID NOS: 2 and 3 are complementary to each other, and have a structure that includes sucA lacking the entire ORE

Then, in order to obtain a sucA gene fragment lacking the internal sequence, equimolar amounts of the aforementioned gene products of the N- and C-terminus sides of sucA were mixed, and used as a template to perform PCR in a conventional manner with the synthetic DNAs of SEQ ID NOS: 5 and 6 as primers and thereby obtain a mutation-introduced sucA gene amplification product. The produced PCR product was purified in a conventional manner and then digested with BamHI, and the resulting DNA was inserted into pBS3, as mentioned above, at the BamHI site. Competent cells of Escherichia coli JM109 (Takara Bio) were transformed with the obtained DNA, plated on an LB plate medium containing 100 μM of IPTG, 40 μg/ml of X-Gal and 25 μg/ml of Km, and cultured overnight, and the white colonies that appeared were selected, and separated into single colonies to obtain transformants. Plasmids were extracted from the obtained transformants, and the plasmid with the target PCR product was designated pBS3ΔsucA.

(1-2) Construction of sucA-deficient Strain

The pBS3ΔsucA obtained in (1-1) mentioned above did not contain any region capable of inducing autonomous replication of the plasmid in cells of coryneform bacteria. Therefore, when coryneform bacteria were transformed with this plasmid, a strain in which this plasmid was incorporated into the chromosome by homologous recombination appeared as a transformant even though it occurred at low frequency. The C. glutamicum ATCC 13869 strain was transformed by the electric pulse method using the plasmid pBS3ΔsucA at a high concentration, applied to the CM-Dex plate medium (5 g/L of glucose, 10 g/L of polypeptone, 10 g/L of yeast extract, 1 g/L of KH₂PO₄, 0.4 g/L of MgSO₄.7H₂O, 0.01 g/L of FeSO₄.7H₂O, 0.01 g/L of MnSO₄.7H₂O, 3 g/L of urea, 1.2 g/L of soybean hydrolysate, 10 μg/L of biotin, 15 g/L of agar, adjusted to pH 7.5 with NaOH) containing 25 μg/ml of kanamycin, and cultured at 31.5° C. for about 30 hours. The strain able to grow on this medium contains the kanamycin resistance gene and the sacB gene originating from the plasmid which had been inserted into the genome as a result of homologous recombination between the sucA gene fragment of the plasmid and that gene on the genome of the ATCC 13869 strain.

Then, these first recombinants were cultured overnight at 31.5° C. in the CM-Dex liquid medium (prepared with the components of the CM-Dex plate medium except for agar) not containing kanamycin, the medium was appropriately diluted and applied to the 10% sucrose-containing Dex-S10 medium (100 g/L of sucrose, 10 g/L of polypeptone, 10 g/L of yeast extract, 1 g/L of KH₂PO₄, 0.4 g/L of MgSO₄.7H₂O, 0.01 g/L of FeSO₄.7H₂O, 0.01 g/L of MnSO₄.4H₂O, 3 g/L of urea, 1.2 g/L of soybean hydrolysate, 10 μg/L of biotin, 15 g/L of agar, adjusted to pH 7.5 with KOH) not containing kanamycin, and culture was performed at 31.5° C. for about 30 hours. As a result, strains were obtained that had become insensitive to sucrose due to elimination of the sacB gene resulting from the second homologous recombination.

The strains obtained as described above included those in which the sucA gene was replaced with that of mutant-type derived from pBS3ΔsucA and those in which the sucA gene reverted to wild-type. Whether the sucA gene is that of mutant-type or wild-type can be easily determined by directly using the cells obtained by the culture on the Dex-S10 plate medium for PCR to detect the sucA gene. Strains that provided a PCR product smaller than that obtained with the chromosomal DNA of the ATCC 13869 strain used as a template in analysis using the primers for PCR amplification of the sucA gene (SEQ ID NOS: 5 and 6) were used as sucA-deficient strains in the following experiments.

L-Glutamic acid-producing ability of the sucA-deficient strains was evaluated by the following method. The strains were cultured on the CM-Dex plate medium, and the grown strains were each cultured at 31.5° C. with shaking in 20 ml of a medium containing 30 g of sucrose, 1 g of KH₂PO₄, 0.4 g of Mg50₄, 15 g of (NH₄)₂50₄, 0.01 g of FeSO₄.7H₂O, 0.01 g of Mn50₄.7H₂O, 13.7 ml of soybean hydrolysate, 200 μg of thiamin hydrochloride, 300 μg of biotin, and 50 g of CaCO₃ in 1 L of pure water (pH was adjusted to 8.0 with KOH) in a Sakaguchi flask. When all glucose in the medium was consumed, the culture was terminated. L-Glutamic acid concentration was measured for the culture supernatant appropriately diluted with water by using Biotech Analyzer (AS-210, Sakura SI). A strain that showed a high L-glutamic acid fermentation yield was selected and designated ATCC13869ΔsucA.

(1-3) Construction of Plasmid for bca Amplification

In order to construct a strain in which expression of the carbonic anhydrase gene (bca) is enhanced, the pVK9 shuttle vector was treated with BamHI, the resulting DNA was ligated with a DNA fragment encoding the enzyme obtained by amplification by PCR using the sequences of SEQ ID NOS: 7 and 8 as primers and the chromosomal DNA of the C. glutamicum ATCC 13869 strain as a template, and then treating with BamHI. Then, the ligation product was used to transform competent cells of Escherichia coli JM109 (Takara Bio), and the cells were applied to the LB plate medium containing 100 μM of IPTG, 40 μg/ml of X-Gal and 25 μg/ml of Cm, and cultured overnight. Then, white colonies that appeared were selected, and separated into single colonies to obtain transformants. Plasmids were extracted from the obtained transformants, and a plasmid in which the bca gene was ligated in the forward direction with respect to the lacZ gene was designated pVK9-bca.

pVK9 is a shuttle vector obtained by blunt-ending the AvaII site of pHSG299 (Takara Bio) and inserting a fragment, which is obtained by excising a region of pHK4 (Japanese Patent Laid-open No. 05-007491) autonomously replicable in coryneform bacteria with BamHI and KpnI, and blunt-ending the region, into the blunt-ended site of pHSG299.

The synthetic DNAs of SEQ ID NOS: 7 and 8 can be designed with reference to the nucleotide sequence of the carbonic anhydrase gene of Corynebacterium glutamicum ATCC 13032 which have been previously reported (GenBank Database Accession No.NC_{—}003450, SEQ ID NO: 13).

(1-4) Introduction of Plasmid for BCA Amplification into ATCC13869ΔsucA Strain

Strains were obtained by transforming the ATCC13869ΔsucA strain with pVK9 (plasmid for control) and pVK9-bca (plasmid for BCA amplification). The transformation was performed by the electric pulse method, and the cells were applied to the CM-Dex plate medium containing 25μg/ml of kanamycin, and cultured at 31.5° C. for about 30 hours to obtain transformants. Strains introduced with each of the aforementioned plasmids were designated ATCC13869ΔsucA(pVK9) and ATCC13869ΔsucA(pVK9-bca), respectively.

2. Confirmation of L-Glutamic Acid Accumulation Improvement Effect in BCA-Enhanced ATCC13869ΔsucA Strain

Effect of the bca amplification was evaluated by using the flask culture evaluation system mentioned in (1-2).

(2-1) Glutamic Acid Accumulation Observed with BCA-Enhanced Strain

Effect of the enhancement of BCA on the improvement of L-glutamic acid accumulation was evaluated with the C. glutamicum ATCC13869ΔsucA(pVK9) strain and the ATCC13869ΔucA(pVK9-bca) strain by culture in the same manner as that used for the evaluation of the sucA-deficient strain described in (1-2) mentioned above. The results obtained after the culture of 12 hours are shown in the following table. It became clear that the L-glutamic acid accumulation amount was higher for the BCA-enhanced strain compared with the control strain (n=4).

TABLE 1

| | L-Glutamic acid accumulation |
|---|---|
| | after culture for 12 hours |
| | (g/L, mean ± standard deviation |
| | % (n = sample number)) |
| ATCC13869ΔsucA(pVK9) | 7.1 ± 0.63 (n = 4) |
| ATCC13869ΔsucA(pVK9-bca) | 8.0 ± 0.78 (n = 4) |

(2-2) L-Amino Acid Accumulation Observed with BCA-Enhanced Strain

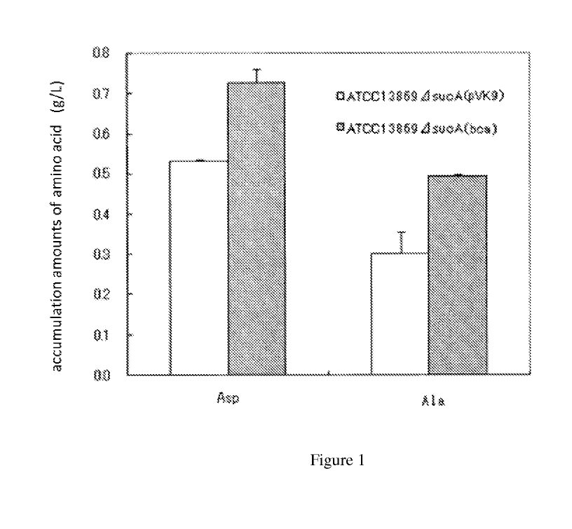

Effect of the enhancement of BCA on improvement of L-amino acid accumulation was evaluated with the C. glutamicum ATCC13869ΔsucA(pVK9) strain and the ATCC13869ΔsucA(pVK9-bca) strain by culture in the same manner as that used for the evaluation of the sucA-deficient strain described in (1-2) mentioned above. One sample for each of culture supernatants obtained after the culture of the two kinds of strains for 24 hours was diluted 51 times with 0.02 N hydrochloric acid, and various L-amino acids in the dilution were quantified with an amino acid analyzer (L-8500, Hitachi Co., Ltd.). The results are shown in FIG. 1. It became clear that accumulation amounts of L-asparatic acid and L-alanine were improved by the enhancement of BCA compared with the control strain (n=2).

While the invention has been described in detail with ereference to preferred embodiments thereof, it will be apparent to one skilled in the art that various changes can be made and equivalents employed, without departing from the scope of the invention. Each of the aforementioned documents are incorporated by reference herein in their entireties.

Explanation of Sequence Listing

SEQ ID NO: 1: Nucleotide sequence of primer for disruption of C. glutamicum ATCC 13869 sucA gene

SEQ ID NO: 2: Nucleotide sequence of primer for disruption of C. glutamicum ATCC 13869 sucA gene

SEQ ID NO: 3: Nucleotide sequence of primer for disruption of C. glutamicum ATCC 13869 sucA gene

SEQ ID NO: 4: Nucleotide sequence of primer for disruption of C. glutamicum ATCC 13869 sucA gene

SEQ ID NO: 5: Nucleotide sequence of primer for amplification of C. glutamicum ATCC 13869 sucA gene

SEQ ID NO: 6: Nucleotide sequence of primer for amplification of C, glutamicum ATCC 13869 sucA gene

SEQ ID NO: 7: Nucleotide sequence of primer for amplification of C, glutamicum bca gene

SEQ ID NO: 8: Nucleotide sequence of primer for amplification of C. glutamicum bca gene

SEQ ID NO: 9: Nucleotide sequence of C. glutamicum ATCC 13869 sucA gene

SEQ ID NO: 10: Amino acid sequence of C. glutamicum ATCC 13869 α-KGDH

SEQ ID NO: 11: Nucleotide sequence of C. glutamicum ATCC 13869 bca gene

SEQ ID NO: 12: Amino acid sequence of C. glutamicum ATCC 13869 Bca

SEQ ID NO: 13: Nucleotide sequence of C. glutamicum ATCC 13032 bca gene

SEQ ID NO: 14: Amino acid sequence of C. glutamicum ATCC 13032 Bca

SEQ ID NO: 15: Nucleotide sequence of Lactobacillus pentosus MD363 xpkA gene

SEQ ID NO: 16: Amino acid sequence of Lactobacillus pentosus MD363 XpkA

SEQ ID NO: 17: Nucleotide sequence of Lactobacillus plantarum xpk1 gene

SEQ ID NO: 18: Amino acid sequence of Lactobacillus plantarum Xpk1

SEQ ID NO: 19: Nucleotide sequence of Bifidobacterium lactis xfp gene

SEQ ID NO: 20: Amino acid sequence of Bifidobacterium lactis Xfp

SEQ ID NO: 21: Nucleotide sequence of C. glutamicum ATCC 13869 pta gene

SEQ ID NO: 22: Amino acid sequence of C. glutamicum ATCC 13869 Pta

SEQ ID NO: 23: Nucleotide sequence of C. glutamicum ATCC 13032 pta gene

SEQ ID NO: 24: Amino acid sequence of C. glutamicum ATCC 13032 Pta

SEQ ID NO: 25: Nucleotide sequence of primer for amplification of C. glutamicum pta gene

SEQ ID NO: 26: Nucleotide sequence of primer for amplification of C. glutamicum pta gene

SEQ ID NO: 27: Nucleotide sequence of E. coli MG1655 yadF gene

SEQ ID NO: 28: Amino acid sequence of E. coli MG1655 YadF

SEQ ID NO: 29: Nucleotide sequence of E. coli MG1655 cynT gene

SEQ ID NO: 30: Amino acid sequence of E. coli MG1655 CynT

1. A method for producing an L-amino acid, which comprises culturing a coryneform bacterium having an L-amino acid-producing ability in a medium to produce and accumulate the L-amino acid in the medium or cells of the bacterium, and collecting the L-amino acid from the medium or cells, wherein said coryneform bacterium has been modified to enhance carbonic anhydrase activity.

2. The method according to claim 1, wherein said carbonic anhydrase activity is enhanced by a method selected from the group consisting of
a) increasing a copy number of a gene encoding carbonic anhydrase,
b) modifying an expression control sequence of the gene, and
c) combinations thereof.

3. The method according to claim 2, wherein the gene encoding the carbonic anhydrase is a DNA selected from the group consisting of:
(a) a DNA comprising the nucleotide sequence of the nucleotide numbers 562 to 1182 of SEQ ID NO: 11, or the nucleotide sequence of SEQ ID NO: 13, and
(b) a DNA that is able to hybridize with a complement of the nucleotide sequence of the nucleotide numbers 562 to 1182 of SEQ ID NO: 11, or the nucleotide sequence of SEQ ID NO: 13, under stringent conditions, and encoding a protein having carbonic anhydrase activity.

4. The method according to claim 1, wherein the bacterium has been further modified to impart D-xylose-5-phosphate phosphoketolase activity and/or fructose-6-phosphte phosphoketolase activity.

5. The method according to claim 1, wherein the bacterium has been further modified to enhance phosphotransacetylase activity.

6. The method according to claim 1, wherein the bacterium has been further modified to enhance pyruvate carboxylase activity.

7. The method according to claim 1, wherein the bacterium has been further modified to enhance phosphoenolpyruvate carboxylase activity.

8. The method according to claim 1, wherein the L-amino acid is selected from the group consisting of L-glutamic acid, L-glutamine, L-proline, L-arginine, L-leucine, and L-cysteine.
